SEQUENCE LISTING (8 sequences)

SEQ ID NO: 1 - DNA, 2209 bp, Citrus sinensis
cgtatttacaaacaaaaatcctgcaacttttgggatcaaagatcccaaatcgaatctttcatttcagttcagttgtcatcacgcgaaacagtaataaaagagtagtacaccaaacaaaggacgaagatgacgttgagtggagtggcacgtgtggcatttgtgggctctcgcaggattttcgcatcacatttgccaagcatgcagagctacactcctgctttctctcgccagatgagtgtggggatgaagagcgttactgaccggcttcgtgattctggccttttacggactcagggtcttattggagggaaatggattgatgcatatgatgggaagactctggaggtccacaatccagcaactggcgaagtgatagcaaatgtatcgtgtatgggaggaaaggaaacaaaagatgctatatcttcggcttttgatgcgtttaattcttggagcaagcttactgctgctgagagaagcaagggtttgcggaaatggtatgatctactgatggctcacaaagaagagcttggccaactaataacattggagcaggggaaacctcttaaagaagctgtcggagaggttgactatggagccaagtttattgagttctatgcagaggaggcgaaacgtgtatatggtgatataattccagcaactctgcctgatcgccggatatttgttctaaagcagcctgtaggtgttgctggtgcaattacaccgtggaactttcccttggctatgattacccgaaaggttggtccggcccttgcttgtggttgtacagtggttctaaaaccctctgaatttacacctctcagtgctttagcagcagctgagctcgccttgcaggctggaatcccaccgggtgttgtgaatgtggttatggggaatgctcctgatattggtgatgctttgctcacgagcccacaggtgaggaagattacatttactggctcaacggctgttggaaagaaactgatggcgggagctgcagggactgttaaaaaggtatctttagagcttggtggcaatgcaccctgcatagtctttgatgatgcagacctgaatgtggcagtcaatggagcgcttgcggcaaagtttcgtaacagtgggcagacctgtgtatgtgcaaacagagtacttgttcaagaaggcatttatgagaaatttgcagatgctttttcaaaagccgtcaagaacctgaaggtgggggatggctttggcgaaggtgtagtccagggtccactaattaataaagctgcattggaaaaggttgaaacttttgtggaagatgctgtttccaagggagcaaaagtccttcttgggggtagaagacatagccttggaatgacattttatgagcctacagtccttagcaatgtcaagagtgagatgcttgtttcgagagaggaagtattcggacctgtggccccacttctatgcttcaaaactgaggaggaagctatccatatggccaatgataccaatgcagggttagctgcttatatatttacaaacaatcttcaacgtacatggcgtgtctctgaagctctagaatatggactcgttggtgttaatgaaggattaatttcaacagaggtggctccttttggtggtgttaaacaatctggtcttggacgggaaggttcaaagtatgggatggatgaattcctggaaataaaatatgtgtgcttgggagacatgaacagaaaatgatgggccagttttgtacctgtatgttaccgggcttatggagaagaaggaaggagcctttagtactgcctttgaactgtattttgaagacggattggaatagtgtgagcctcaagcgtttgatgtggaggtagtagtgagccatagttggtttgctattgataccccatgtacataaataagtttgcaaaatggaacttgaatgtctagtagtgagccataggtggtttgctattgataccccatgtacataaataagtttgcaaaatggaacttgaatgtctggtgtcataaagattttgccgtcaacacccaaaacctaatgggaaagaacttttcttcttttggtatcattacatttccgcggggttatgtttctcttgatcagatatgaatcaagattccatggtatttatacccccccatctttctgtaacagtgttgcttcagtactctctttgcataacaatttgtaaaggttttgttggtggaa

SEQ ID NO: 2 - protein, 535 aa, Citrus sinensis
MTLSGVARVAFVGSRRIFASHLPSMQSYTPAFSRQGIVCQMSVGMKSVTDRLRDSGLLRTQGLIGGKWIDAYDGKTLEVHNPATGEVIANVSCMGGKETKDAISSAFDAFNSWSKLTAAERSKGLRKWYDLLMAHKEELGQLITLEQGKPLKEAVGEVDYGAKFIEFYAEEAKRVYGDIIPATLPDRRIFVLKQPVGVAGAITPWNFPLAMITRKVGPALACGCTVVLKPSEFTPLSALAAAELALQAGIPPGVVNVVMGNAPDIGDALLTSPQVRKITFTGSTAVGKKLMAGAAGTVKKVSLELGGNAPCIVFDDADLNVAVNGALAAKFRNSGQTCVCANRVLVQEGIYEKFADAFSKAVKNLKVGDGFGEGVVQGPLINKAALEKVETFVEDAVSKGAKVLLGGRRHSLGMTFYEPTVLSNVKSEMLVSREEVFGPVAPLLCFKTEEEAIHMANDTNAGLAAYIFTNNLQRTWRVSEALEYGLVGVNEGLISTEVAPFGGVKQSGLGREGSKYGMDEFLEIKYVCLGDMNRK

SEQ ID NO: 3 - DNA, 2130 bp, Arabidopsis thaliana
gacggatccaattttgaagatattttacttgtaaaaaatctcttgacaacatataatttcgttgcctagtagcttcttcacagcttctgatctcactagtgttcgtcatctttgttcgattgaagtttgggaagtggtgatggtaataggagcagcagcgcgtgtagccatcggcggttgtcgaaagctcatctcgtcacatacctccctcctcctcgtctcttctcagtgtcgccagatgagtatggacgcacaaagcgtttctgaaaagcttaggagttctggtctattacgaactcaaggtctaattggaggcaagtggcttgactcgtatgacaataagacaatcaaggttaacaatccagcaaccggtgaaattatagctgatgttgcatgtatgggaacaaaagagacaaatgatgctattgcttcttcttatgaagcatttacttcttggagcagattgactgctggagagaggagtaaagttttacggagatggtatgacctcctgattgcacacaaggaagaacttggacaacttataactttggagcaaggaaaaccactcaaggaagccatcggagaggtagcatatggggcaagttttattgagtactatgcggaggaggcaaaacgtgtatatggtgatataattcctccgaatctgtctgatcgccgactgttggttctaaaacagcctgtcggtgttgttggtgcaattaccccttggaacttccccttagccatgattactcggaaggtcggccctgctcttgcttctggatgcacggtggttgttaaaccatctgaacttacgcccctaacagcacttgctgcggctgaacttgcacttcaagctggagttcctccgggggcacttaatgtggtcatgggaaatgctcctgaaattggggatgctttgcttacgagtccacaggtgagaaaaatcacgttcacgggatcaacagcagttgggaagaagttgatggcagctgctgcacctaccgtcaagaaggtttctctagaacttggcggcaacgcaccctctatagtatttgatgatgcagacctggatgtagctgtaaaaggaacgcttgcagcgaaatttaggaatagtggtcagacatgtgtttgtgcgaacagagtacttgtgcaagatggtatctatgataaatttgctgaggccttttctgaagcggttcaaaaattagaagtaggagatgggtttagggatgggacaacccagggtccacttataaatgatgcagcagtgcaaaaggtagagacatttgtacaagatgctgtttctaagggagcaaaaatcatcattggtggcaaaaggcacagtctagggatgactttctatgagcctactgttatccgcgatgtttcggataacatgatcatgtctaaggaggagatttttggacctgtagctccccttattcggttcaaaaccgaggaggacgctatcagaattgctaatgacacaattgcaggacttgctgcttatatattcacaaacagtgtccaaagatcttggcgtgtatttgaagcacttgaatatggacttgtaggggtgaacgaaggactcatatcaacagaggtggctccattcgggggagtgaagcagtctggtcttggaagggaaggatccaagtatggtatggacgaataccttgagatcaaatacgtatgcttgggagatatgaatagacactgatttggtttgtttgaaaagctttagaaattgtaagtttttctcctccactcgtatccttctaataaaagcttactgtggaacataataaggattcggaggatacttcttaagaaataaagatttggtctacgaccaattgttagcatgatttttaagtaataatttggggtttccattttatttatctttgtttacgttatttttttctttgttttacgttaatttttgtaattcggacaatttttgctttggatatacccaaaaaaataaaaaaataaacgttgttgttctttgcttcatttatcagcaatttttagaaatcgattgggtttatttttcttttttcatcagtatcttgtcattactatttataaacgctttatgtctcatgtgacaatgttttg

SEQ ID NO: 4 - protein, 528 aa, Arabidopsis thaliana
MVIGAAARVAIGGCRKLISSHTSLLLVSSQCRQMSMDAQSVSEKLRSSGLLRTQGLIGGKWLDSYDNKTIKVNNPATGEIIADVACMGTKETNDAIASSYEAFTSWSRLTAGERSKVLRRWYDLLIAHKEELGQLITLEQGKPLKEAIGEVAYGASFIEYYAEEAKRVYGDIIPPNLSDRRLLVLKQPVGVVGAITPWNFPLAMITRKVGPALASGCTVVVKPSELTPLTALAAAELALQAGVPPGALNVVMGNAPEIGDALLTSPQVRKITFTGSTAVGKKLMAAAAPTVKKVSLELGGNAPSIVFDDADLDVAVKGTLAAKFRNSGQTCVCANRVLVQDGIYDKFAEAFSEAVQKLEVGDGFRDGTTQGPLINDAAVQKVETFVQDAVSKGAKIIIGGKRHSLGMTFYEPTVIRDVSDNMIMSKEEIFGPVAPLIRFKTEEDAIRIANDTIAGLAAYIFTNSVQRSWRVFEALEYGLVGVNEGLISTEVAPFGGVKQSGLGREGSKYGMDEYLEIKYVCLGDMNRH

SEQ ID NO: 5 - DNA, 104 bp, Arabidopsis thaliana
gatgtctataaatataagagaccctcttatagtaagcagagttgttggagacgttcttgatccgtttaatagatcaatcactctaaaggttacttatggccaaa

SEQ ID NO: 6 - DNA, 590 bp, Citrus paradisi
aaaaatcctgcaacttttgggatcaaagatcccaaatcgaatctttcatttcagttcagttgtcatcacgcgaaacagtaataaaagagtagtacaccaaacaaaggacgaagatgacgttgagtggagtggcacgtgtggcatttgtgggctctcgcaggattttcgcatcacatttgccaagcatgcagagctacactcctgctttctctcgccagatgagtgtggggatgaagagcgttactgaccggcttcgtgattctggccttttacggactcagggtcttattggagggaaatggattgatgcatatgatgggaagactctggaggtccacaatccagcaactggcgaagtgatagcaaatgtatcgtgtatgggaggaaaggaaacaaaagatgctatatcttcggcttttgatgcgtttaattcttggagcaagctcactgctgctgagagaagcaagggtttgcggaaatggtatgatcaactgatggctcacaaagaagagcttggccaactaataacattggagcaggagaaacctcttaaagaagctgtcggtgaggttgactatggagccaagttt

SEQ ID NO: 7 - DNA, 584 bp, Unknown
  Citrus sinensis cv. Hamlin
tcctgcaacttttgggatcaaagatcccaaatcgaatctttcatttcagttcagttgtcatcacgcgaaacagtaataaaagagtagtacaccaaacaaaggacgaagatgacgttgagtggagtggcacgtgtggcatttgtgggctctcgcaggattttcgcatcacatttgccaagcatgcagagctacactcctgctttctctcgccagatgagtgtggggatgaagagcgttactgaccggcttcgtgattctggccttttacggactcagggtcttattggagggaaatggattgatgcatatgatgggaagactctggaggtccacaatccagcaactggcgaagtgatagcaaatgtatcgtgtatgggaggaaaggaaacaaaagatgctatatcttcggcttttgatgcgtttaattcttggagcaagcttactgctgctgagagaagcaagggtttgcggaaatggtatgatctactgatggctcacaaagaagagcttggccaactaataacattggagcaggggaaacctcttaaagaagctgtcggagaggttgactatggagccaagtt

SEQ ID NO: 8 - DNA, 625 bp, Unknown
  Citrus sinensis  X Poncirius trifoliata
tcgtatttaccaacaaaaatcctgcaacttttgggatcaaagatcccaaatcgaatctttcatttcatttgggttcttgtgctcagcttcggttcagttgtcatcacctgaaacagtaataaaagattagtacacgaaacaaaggacgaagatgacgttgagtggagtggcacgtgtggcatttgtgggctctctcaggattttcgcatcacatttgccaagcatgcagagctacactcctgctttctctcgccagatgagtgtggggatgaagagcgttactgaccggcttcgtgattctggccttttacggactcagggtcttattggagggaaatggattgatgcatatgatgggaagactctggaggtccacaatccagcaactggcgaagtgatagctaatgtatcgtgtatgggaggaaaggaaacaaaagatgctatatcttcggcttttgatgcgtttaattcctggagcaagctcactgccgctgagagaagcaagggtttgcggaaatggtatgatctactgatggctcacaaagaagagcttggccaactaataacattggagcaggggaaacctcttaaagaagctgtcggtgaggttgactatggagccaag

(19) United States
(12) Patent Application Publication
(10) Pub. No.: US 2021/0317472 A1
(43) Pub. Date: Oct. 14, 2021

(54) METHODS AND COMPOSITIONS USING RNAi FOR PREVENTING OR REDUCING INFECTIONS OF CROP PLANTS BY BIOTROPHIC PATHOGENS

(71) Applicants: Integrated Plant Genetics, Inc., Gainesville, FL (US); University of Florida Research Foundation, Inc., Gainesville, FL (US)

(72) Inventors: Tina STRAUSS, Gainesville, FL (US); Dean W. GABRIEL, Gainesville, FL (US)

(21) Appl. No.: 17/271,938

(22) PCT Filed: Aug. 29, 2019
(86) PCT No.: PCT/US2019/048870

§ 371 Date: Feb. 26, 2021

Related U.S. Application Data
(60) Provisional application No. 62/724,470, filed on Aug. 29, 2018.

(51) Int. Cl. C12N 15/82

(52) U.S. Cl. CPC C12N 15/8286; C12N 15/8218

(57) ABSTRACT

The present invention teaches methods and compositions useful for treating, preventing, or curing pathogen infections of living plants. In particular, the present invention teaches methods of enhancing plant response to pathogen-associated molecular patterns using RNA interference of plant genes. The methods and compositions described herein are effective at treating biotrophic pathogens, including Liberibacters.

### CROSS-REFERENCE TO RELATED APPLICATIONS

The present application claims priority to, and the benefits of United States Provisional Patent Application No. 62/724,470, filed on Aug. 29, 2018, which is incorporated herein by reference in its entirety.

### GOVERNMENT SUPPORT

This invention was made with government support under Cooperative Agreement 17-8130-0640-CA and AP18PPQS&T00C131 awarded by the United States Department of Agriculture. The government has certain rights in the invention.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: INTE_016_1WO_SeqList_ST25.txt, date created: Aug. 28, 2019, file size≈18 kilobytes)

### FIELD OF THE INVENTION

The present disclosure relates to methods and compositions for preventing, eliminating, reducing, or otherwise ameliorating infections and/or damage of crop plants by biotrophic bacterial and fungal pathogens.

### BACKGROUND OF THE INVENTION

All animal and plant cells have a highly regulated cell suicide program designed to limit the damage done to one cell or a group of cells from affecting the entire organism. This is why cells die after radiation damage from sunburn, for example; otherwise, the radiation damage would result in mutations that might result in cancers, or in skin tissue with greatly aged appearance and performance. This suicide program is tightly controlled in all organisms, and it requires a combination of factors to come together to trigger the cell death program. Once initiated, it is irreversible.

Some pathogens have evolved mechanisms to avoid triggering cell death programs, thus circumventing an important plant defense. A solution for regaining control of cell death defense mechanisms is needed to address the emergence and proliferation of and/or damages caused by these pathogens.

### SUMMARY OF THE INVENTION

The present disclosure teaches compositions and methods useful for protecting plants against both intracellular and intercellular bacterial and fungal attack, growth and infection, comprising the silencing of SSADH genes.

In some aspects, the present disclosure is drawn to a recombinant nucleic acid molecule comprising a nucleic acid sequence comprising at least 15, 16, 17, 18, 19, 20, 21, 22, 23, 24, or 25 contiguous nucleotides complementary to SEQ ID NO: 1, SEQ ID NO: 3, SEQ ID NO: 6, SEQ ID NO: 7 or SEQ ID NO: 8. In some embodiments, the term recombinant nucleic acid molecule is a synthetic molecule. In some embodiments, transcription of the nucleic acid molecule or the presence of the nucleic acid molecule reduces expression of a succinic semialdehyde dehydrogenase (SSADH) gene in a plant cell.

In some embodiments, the recombinant nucleic acid molecule is a DNA molecule. In some embodiments, the DNA molecule is an inverted repeat. In some embodiments, the inverted repeat comprises a first fragment comprising at least 15, 16, 17, 18, 19, 20, 21, 22, 23, 24, or 25 contiguous nucleotides of SEQ ID NO: 1, SEQ ID NO: 3, SEQ ID NO: 6, SEQ ID NO: 7 or SEQ ID NO: 8, and a second fragment comprising at least 15, 16, 17, 18, 19, 20, 21, 22, 23, 24, or 25 contiguous nucleotides complementary to SEQ ID NO: 1, SEQ ID NO: 3, SEQ ID NO: 6, SEQ ID NO: 7 or SEQ ID NO: 8, and a RNA molecule transcribed from said DNA molecule anneal to each other perfectly or partially. In some embodiments, the first fragment and the second fragment is linked through a loop sequence, so a RNA molecule transcribed from said DNA molecule form a hairpin dsRNA or a stem-loop dsRNA. In some aspects, the recombinant nucleic acid molecule is operably linked to a GUS loop sequence or an intron sequence.

In some embodiments, the recombinant nucleic acid molecule is an DNA molecule, and the recombinant nucleic acid molecule comprises to a promoter operably linked to the contiguous nucleotides. In some embodiments, the promoter is a heterologous promoter to the recombinant nucleic acid molecule. In some embodiments, the promoter is suitable for gene expression in a bacterial species. In some embodiments, the promoter is suitable for gene expression in a plant species. In some embodiments, transcription of the nucleic acid molecule reduces expression of an SSADH gene in a plant cell.

In some embodiments, the nucleic acid molecule is a DNA molecule, and transcription of the nucleic acid molecule reduces expression of an SSADH gene in a plant cell, a plant part, a plant, or a plant population. In some embodiments, the reduced expression of an SSADH gene in the plant cell increases resistance to a pathogen in the plant cell, the plant part, the plant, or the plant population. In some embodiments, transcription of the nucleic acid sequence produces an interfering RNA molecule targeting the SSADH gene, wherein the interfering RNA is a double-stranded RNA or antisense RNA. In some embodiments, one strand of the double-stranded RNA or the antisense RNA comprises at least 15, 16, 17, 18, 19, 20, 21, 22, 23, 24 or 25 contiguous nucleotides that are complementary to SEQ ID NO: 1, SEQ ID NO: 3, SEQ ID NO: 6, SEQ ID NO: 7 or SEQ ID NO: 8.

In some embodiments, the nucleic acid molecule is a RNA molecule. In some embodiments, the RNA molecule is an interfering RNA molecule targeting the SSADH gene. In some embodiments, the interfering RNA is a double-stranded RNA, a siRNA, or an antisense RNA. In some embodiments, one strand of the double-stranded RNA, the siRNA, or the antisense RNA comprises at least 15, 16, 17, 18, 19, 20, 21, 22, 23, 24 or 25 contiguous nucleotides that are complementary to SEQ ID NO: 1, SEQ ID NO: 3, SEQ ID NO: 6, SEQ ID NO: 7 or SEQ ID NO: 8. In some embodiments, presence of the RNA molecule in a plant cell reduces expression of SSADH gene in the plant cell, or plant cells within the same plant part, or same plant.

In some aspects, the present disclosure is drawn to a recombinant nucleic acid molecule comprising a nucleic acid sequence comprising at least 15, 16, 17, 18, 19, 20, 21, 22, 23, 24, or 25 contiguous nucleotides of a nucleic acid sequence sharing at least 85%, 86%, 87%, 88%, 89%, 90%, 91%, 92%, 93%, 94%, 95%, 96%, 97%, 98%, 99%, or more sequence identity with a sequence that is complementary to SEQ ID NO: 1, SEQ ID NO: 3, SEQ ID NO: 6, SEQ ID NO: 7 or SEQ ID NO: 8.

In some aspects, the recombinant nucleic acid molecule is operably linked to a movement leader sequence. In some aspects, the movement leader sequence comprises at least 18 continuous nucleotides of SEQ ID NO:5, or corresponding RNA sequence.

In some aspects, the present disclosure is drawn to a recombinant vector comprising the recombinant nucleic acid molecule. In some aspects, the present disclosure is drawn to a transgenic plant cell comprising the recombinant nucleic acid molecule and/or the recombinant vector. In some aspects, the present disclosure is drawn to a transgenic plant comprising the recombinant nucleic acid molecule and/or the recombinant vector. In some aspects, the present disclosure is drawn to a transgenic plant part comprising the recombinant nucleic acid molecule and/or the recombinant vector. In some aspects, the present disclosure is drawn to a transgenic seed comprising the recombinant nucleic acid molecule and/or the recombinant vector. 12.

In some aspects, the present disclosure is drawn to a plant comprising a no transgenic grafted scion, and a transgenic rootstock. In some aspects, the rootstock comprise a recombinant nucleic acid molecule of the present disclosure.

In some aspects, the present disclosure is drawn to a composition comprising a recombinant nucleic acid molecule of the present disclosure. In some embodiments, the recombinant nucleic acid molecule is a DNA molecule. In some embodiments, the recombinant nucleic acid molecule is an RNA molecule. In some embodiments, the recombinant nucleic acid molecule is an RNA molecule, and the composition is for delivering the RNA molecule into a plant. In some embodiments, the composition is for delivering the RNA molecule into phloem cells of a plant. In some embodiments, the composition is in a nanoemulsion formulation. In some embodiments, the composition is for down-regulating a SSADH gene in a plant cell. In some embodiments, the composition is for repressing, preventing, or otherwise reducing a bacterial or fungal infection of a plant.

In some aspects, the present disclosure is drawn to a method of repressing, preventing, or otherwise reducing a bacterial or fungal infection of a plant. In some aspects, the method comprises expressing the recombinant nucleic acid and/or the vector in a plant, a cell thereof, or a part thereof. In some aspects, the plant cell is a citrus tree plant cell. In some aspects, the infection is caused by a biotrophic plant pathogen. In some aspects, the infection is caused by a Liberibacter. In some aspects, the infection is caused by a citrus-infecting Liberibacter. In some aspects, the infection is caused by Ca. Liberibacter asiaticus (Las).

In some aspects, the present disclosure is drawn to a method of repressing, preventing, or otherwise reducing a bacterial or fungal infection of a plant. In some aspects, the method comprises topical application, injection, or any known method of administering the nucleic acid molecule to a plant. In some aspects, the topical application comprises a composition that comprises lecithin and/or gelatin. In some aspects, the composition that comprises the lecithin and/or gelatin is an emulsion. In some aspects, the plant is a citrus tree. In some aspects, the infection is caused by a biotrophic plant pathogen. In some aspects the infection is caused by a Liberibacter. In some aspects, the infection is caused by a Ca. Liberibacter asiaticus (Las).

In some aspects, the present disclosure is drawn to a method of identifying a functional homolog equivalent to SEQ ID NO:1 or SEQ ID NO:2 in other plant species.

The present disclosure relates to methods and compositions for preventing, reducing, eliminating or otherwise ameliorating infections and/or damage of crop plants by bacterial and fungal pathogens. The percentage reduction in pathogen infection and/or plant damage for plants protected using the compositions and methods of the present invention is at least 5%, 10%, 15%, 20%, 25%, 30%, 35%, 40%, 45%, 50%, 55%, 60%, 65%, 70%, 75%, 80%, 85%, 90%, 95%, 96%, 97%, 98% or 99% greater/better when compared to an appropriate control or check plant grown under the same plant husbandry conditions. The amount of pathogen infection and/or plant damage can be measured using methods well known to those skilled in the art. Plant infection, e.g., can be measured as the percentage of necrotic tissue on the plants. Plant damage. e.g., can be measured as total yield of a specific plant part (e.g., number or weight of seeds, number or weight of pods, plant weight, plant height, number of weight of flowers, root mass measured in volume or by weight, etc.). An appropriate control or check plant is one in which the SSADH gene(s) have not been silenced as they are silenced in the test plant(s) according to the compositions and methods of the present invention.

Specifically, the disclosure teaches use of ribonucleic acid (RNA) interference (RNAi), double stranded RNA (dsRNA), and/or anti-sense RNA (aRNA or asRNA) for their potential to increase the plant apoptotic response to pathogens by increasing the Reactive Oxygen Response levels to plant pathogens. More specifically, the disclosure teaches the gene specific targeting of SSADH homologs to control biotrophic pathogens, particularly, but not limited to, those caused by Liberibacters.

In one embodiment, the specific target is the citrus succinate-semialdehyde dehydrogenase mitochondrial isoform X1 homolog (SSADH) of Citrus sinensis cultivar Valencia (SEQ ID NO: 1). In another embodiment, the SSADH homolog is from Clementine tangerine, Citrus×clementina. In another embodiment, the grapefruit homolog is utilized. In another embodiment, the Hamlin homolog of Citrus sinensis (sweet orange) cultivar Hamlin is used.

In another embodiment, the specific target was from tomato, potato, tobacco, celery, pear, apple, plum, cherry, olive, or Vitis vinifera grapes.

In further embodiments, any segment, section or part of the full length sweet orange genome SSADH mRNA homolog, GenBank XM 006493686.2 can be used, including both the 5′ and 3′ untranslated regions (i.e., not only the fragments currently deposited in GenBank). For example, when comparing a 497 bp experimentally derived sequence from Hamlin sweet orange to the same 497 bp experimentally derived sequence from Carrizo rootstock, the sequences were about 97% identical. When the same Hamlin and Carrizo sequences were compared to the equivalent region of Valencia orange, they were about 98.2% and about 97.6% identical, respectively. Indeed, any SSADH homolog, including the 5′ and 3′ untranslated regions found in any citrus host could be used by those skilled in the art for the purpose of silencing the citrus SSADH gene, since it is well known that the untranslated regions of mRNA can serve as excellent targets of siRNAs (Deng et al 2012; Lai et al 2013). Thus in some embodiments, the polymerase chain reaction (PCR) cloning strategy as described herein from Carrizo, Hamlin, or Valencia or any other citrus source is useful for identification of a SSADH homolog useful for silencing of the SSADH genes in any citrus host, including any species of the genus citrus. Citrus is a genus of flowering trees and shrubs in the rue family, Rutaceae. Plants in the genus produce citrus fruits, including important crops like oranges, lemons, grapefruit, pomelo and limes.

Given the current and growing availability of genomic DNAs, multiple corresponding SSADH genes can now readily be identified by those skilled in the art from virtually any plant source for which a DNA sequence is available using a PCR cloning strategy similar to that taught here, including SSADH genes from citrus and other woody species such as Malus domestica apple, Theobroma cacao cocoa, Prunus persica peach, Populus deltoides poplar, Olea europaea olive, vines such as Vitis vinifera grape, and agronomic crop plants such as Gossypium hirsutum cotton, Glycine max soybean. Arabidopsis, Solanum tuberosum potato, Solanum lycopersicum tomato, Nicotiana tobacco and many others. In further embodiments, any segment, section or part of the full length genomic SSADH mRNA homologs from any of these species can be used to silence the SSADH genes of varieties of that species, including both the 5′ and 3′ untranslated regions (i.e., not only the fragments currently deposited in GenBank).

The present invention also provides compositions and methods for the protection and/or curing of plants from infections caused by biotrophic bacteria and fungi by complete or partial (i.e., incomplete) suppression of SSADH homologs. In one embodiment, the invention provides compositions and methods for the protection of citrus cells from infection by biotrophs. In some embodiments, the invention provides compositions and methods for the protection and curing of phloem cells of citrus, solanaceous, and other plants families infected by various species of the bacterial pathogen genus Liberibacter. In some embodiments, the invention provides compositions and methods for the protection and curing of citrus phloem from infection by Ca. Liberibacter asiaticus (Las).

The present invention also provides compositions and methods for the protection of grafted scions that are nontransgenic by the use of rootstock for which one or more SSADH genes have been silenced or attenuated using the methods of the present invention.

In a preferred embodiment, the present invention provides compositions and methods for the protection and curing of nontransgenic citrus trees that are infected by using a polynucleotide spray, including, e.g., via a double stranded RNA (dsRNA) spray that can transiently suppress SSADH homolog expression. When applied as a dsRNA spray to plants, the silencing effect on the target gene occurs within 2-3 days, and the effect lasts for 3-4 weeks (de Andrade & Hunter, 2016). The effect of dsRNA applications is therefore transient. In some embodiments, the present invention provides compositions and methods for the protection and curing of citrus phloem cells from infection by Liberibacters, including Las, or citrus mesophyl cells attacked by X. citri.

In some embodiments, the present invention teaches that dsRNA about 10, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 base pairs in length or as short fragments of about base pairs in length, can induce RNAi and suppress SSADH homolog expression in citrus when applied as sprays or as soil drench from the outside of the citrus plant. In some embodiments, the RNAi can be induced not simply by application of dsRNA, but by any double stranded polynucleotide, including synthetic polynucleotides. In some embodiments, antisense polynucleotides can be formed not simply from polynucleotides, but also from phosphorodiamidate morpholino oligomers (PMOs).

In some embodiments, the RNAi can travel across the graft union from transgenic rootstock to nontransgenic scion, thus obviating the need for scion transformation as well as reducing the regulatory burden attendant to marketing transgenic plants that may shed transgenic pollen and or marketing transgenic fruit. In addition, an RNA movement leader can be used that confers the capacity to move an antisense RNA (aRNA) or dsRNA long distances in plants, including but not limited to across a graft union from transgenic rootstock to nontransgenic scions. A potential movement leader sequence from Arabidopsis thaliana, flowering locus T (FT) from nucleotide position 326 to nucleotide 429 in GenBank: GQ395494.1 (SEQ ID NO: 5) can be transcriptionally fused in the sense orientation at the 5′ end of the dsRNA, and/or in the antisense orientation at the 3′ end of the synthetic dsRNA.

The present invention also provides silencing constructs based on phloem specific gene expression that: 1) result in keeping the engineered silencing limited to phloem only, and 2) allows spread from phloem to mesophyl and epidermal cells.

In some embodiments, the present invention teaches compositions and methods for delivering dsRNA into phloem cells using nanoemulsions that stabilize the dsRNA, which is then effective in repressing, preventing or otherwise reducing bacterial or fungal infections of a plant comprising expressing an antisense or RNA interference construct based on a SSADH protein or nucleic acid sequence.

In some embodiments the present invention teaches methods of identifying SSADH family genes through the use of SSADH from citrus, Arabidopsis or any other source and/or through use of SEQ ID NO: 1 or SEQ ID NO: 3 or a protein or protein fragment encoded by an amino acid sequence having at least 70%, or at least 80%, or at least 90%, or at least 95%, or at least 98% amino acid sequence similarity to the predicted peptide sequence encoded by SEQ ID NO: 2 or SEQ ID NO: 4. In certain embodiments, the present invention teaches an isolated nucleic acid molecule coding for at least 15, 16, 17, 18, 19, 20, 21, 22, 23, 24, or 25 contiguous bases pairs of homology to SSADH from citrus, Arabidopsis or any other source, and/or SEQ ID NO: 1, SEQ ID NO: 3, SEQ ID NO: 6, SEQ ID NO: 7 or SEQ ID NO: 8 that can be used for controlling plant diseases. In other embodiments, the methods and compositions of the present invention can be used to treat, reduce, or eliminate disease in citrus plants.

In some embodiments, the present invention teaches the complete or partial (i.e., incomplete) down-regulation of a SSADH gene to treat diseases caused by biotrophic plant pathogens. In some embodiments, the disease treated by the methods and compositions of the present invention is caused by a Liberibacter. In some embodiments, the disease treated by the methods and compositions of the present invention is caused by a Liberibacter infecting a citrus plant. In certain embodiments, the Liberibacter of the present invention is Ca. Liberibacter asiaticus (Las).

In some embodiments, the present invention teaches that the isolated nucleic acid molecules of the present invention are operably-linked to a nucleic acid molecule coding for an endoplasmic reticulum (ER) retention signal sequence.

In some embodiments, the present invention teaches that the isolated nucleic acid molecules of the present invention are operably-linked to a nucleic acid molecule coding for one or more expression control elements. In some embodiments, the nucleic acids of the present invention are expressed using a CaMV 35S or an AtSuc2 promoter. In some embodiments, the present invention teaches vectors for expressing the nucleic acids taught by the present invention.

In some embodiments the present invention teaches a host cell transformed to contain the at least one of the nucleic acid molecules of the present invention. In other embodiments, the host cell of the present invention is a eukaryotic or prokaryotic host cell.

In some embodiments, the present provides methods of enhancing a plant's immune response to infection, said method comprising complete or partial (i.e., incomplete) down-regulating the expression of a SSADH gene wherein said plant has increased NDR1 expression in response to “pathogen associated molecular patterns” (PAMPs) compared to a control plant with unaltered SSADH gene expression.

In some embodiments, the present disclosure teaches the down-regulation of one or more SSADH genes with at least 70%, or at least 80%, or at least 90%, or at least 95%, or at least 98% sequence identity to SEQ ID NO: 1, SEQ ID NO: 3, SEQ ID NO: 6, SEQ ID NO: 7 or SEQ ID NO: 8. In some embodiments, the present invention teaches the use of antisense RNA for down-regulating SSADH genes. In some embodiments, the present invention teaches the use of RNAi for down-regulating SSADH genes. The RNAi can be achieved in multiple ways. In some embodiments, the RNAi is achieved by topical spray application of dsRNAs ranging in size from about 200 to about 2,000 bp in length (“long dsRNAs”). In some embodiments, the RNAi is achieved by topical spray applications of dsRNAs predigested to about 15 to 30 bp in size, also known as small interfering RNAs (“siRNAs”). In some embodiments, the RNAi is achieved by topical spray applications of dsRNAs predigested to ca. 23 bp in size, also known as siRNAs. In some embodiments, the RNAi is achieved by either siRNAs or long dsRNAs applied by laser etching or mechanical penetration of leaf and stem cuticle layers. In some embodiments, the RNAi is achieved by either siRNAs or long dsRNAs applied by root applications, by soil drench and/or by direct trunk injections.

In some embodiments, the methods of the present invention increase plant resistance to at least one biotrophic pathogen. In certain embodiments, the biotrophic pathogens of the present invention are Liberibacters.

In some embodiments, the present invention teaches a recombinant or transgenic plant, or part thereof, comprising a construct comprising a polynucleotide capable of triggering RNA interference and down-regulating a SSADH gene with at least 70%, or at least 80%, or at least 90%, or at least 95%, or at least 98% sequence identity to SEQ ID NO: 1, SEQ ID NO: 3, SEQ ID NO: 6, SEQ ID NO: 7 or SEQ ID NO: 8, wherein said recombinant plant exhibits enhanced response to PAMP triggers. In other embodiments, the present disclosure provides transgenic plant cells, transgenic seeds, and progeny plants, and parts thereof, derived from transgenic plants, comprising a recombinant nucleic acid construct comprising a nucleic acid molecule comprising at least 15, 16, 17, 18, 19, 20, 21, 22, 23, 24, or 25 contiguous nucleotides from SEQ ID NO: 1, SEQ ID NO: 3, SEQ ID NO: 6, SEQ ID NO: 7 or SEQ ID NO: 8 operably linked to a heterologous promoter, that when transcribed reduces expression of a SSADH gene in a plant. In some embodiments, the present invention teaches a recombinant plant comprising a construct comprising a polynucleotide capable of triggering RNAi and down-regulating a SSADH gene with 70%, or at least 80%, or at least 90%, or at least 95%, or at least 98% sequence identity to SEQ ID NO: 1, SEQ ID NO: 3, SEQ ID NO: 6, SEQ ID NO: 7 or SEQ ID NO: 8 wherein said recombinant plant exhibits enhanced resistance to Liberibacters and confers said resistance long distances, including across the graft union to nontransgenic scions. In some embodiments, the present invention teaches a topically applied spray formulation comprising a polynucleotide capable of triggering RNA interference in nontransgenic plants and down-regulating a SSADH gene with at least 70%, or at least 80%, or at least 90%, or at least 95%, or at least 98% sequence identity to SEQ ID NO: 1, SEQ ID NO: 3, SEQ ID NO: 6, SEQ ID NO: 7 or SEQ ID NO: 8, wherein said spray formulation confers enhanced resistance to Liberibacters and confers said resistance long distances in the plant. In some embodiments, the present invention teaches a topically applied root application or soil drench formulation comprising a polynucleotide capable of triggering RNAi in nontransgenic plants and down-regulating a SSADH gene with at least 70%, or at least 80%, or at least 90%, or at least 95%, or at least 98% sequence identity to SEQ ID NO: 1 or SEQ ID NO:3, wherein said spray formulation confers enhanced resistance to Liberibacters and confers said resistance long distances throughout the plant. In other embodiments, the present invention teaches a grafted plant with recombinant rootstock and an untransformed scion.

### BRIEF DESCRIPTION OF THE DRAWINGS

FIG. 1. SSADH gene suppression in C. sinensis sweet orange cut seedling assay using dsRNA from the SSADH mRNA sequence. Four inch cut seedlings of Citrus sinensis cv. Madam vinous were placed in an aqueous solution containing either 5 ng or 100 ng of commercially prepared dsRNA (500 nt in length) designed from SEQ ID NO. 1. qRT-PCR was used to determine levels of expression of SSADH in all samples, using primers well outside of the region of SSADH used for the RNA treatments. qRT-PCR levels were normalized against 4 control seedlings (set at 100%), and relative expression levels of SSADH using 4 plants of water controls and 4 plants each in both treatments levels. SSADH dsRNA moved long distances up the stem and into the leaves within 3 days, and was effective at 5 ng/4 inch stem (weighing 150-300 mg).

FIG. 2. Ca. Liberibacter asiaticus (Las) infection is reduced in commercially grown citrus after spray treatment using long (500 nt) SSADH dsRNA formulated into nanoemulsion 224. An RNA-based, live cell assay was performed in four year old, commercially grown grapefruit trees in a field trial. All trees were 100% infected with Las. Control plants were mock sprayed with water. qRT-PCR was used as in FIG. 1 to determine levels of expression of Las GroEL. Plant cox1 was used to normalize expression. GroEL gene expression is useful as an indicator of actively growing (live) Las cells, and this Las gene expression was significantly (P<0.05%) suppressed after a single application of SSADH dsRNA in formula 224 applied to field grown, commercial grapefruits, one-month post treatment.

FIG. 3. Las gene expression (groEL) live cell assay for long distance effect in C. paradisi (grapefruit) after two SSADH dsRNA treatments applied by laser etching in a relatively narrow band on one side of the treated trees (5 year old, heavily Las-infected grapefruits in a commercial grove). Treatments not sharing the same letter are significantly different by Tuckey's.

FIG. 4. Las gene expression (groEL) live cell assay for long distance effect in C. pradisi (grapefruit) in three independent field experiments, after a single treatment with either SSADH (in two of the experiments) or a combination of SSADH plus BAG6 dsRNA applied by laser etching in a relatively narrow band on one side of the treated trees (5 year old, heavily Las-infected grapefruits in a commercial grove).

FIG. 5. SSADH dsRNA applied by root uptake suppressed sweet orange SSADH expression in leaves. SSADH expression in leaves over 2 a week period, after applying 20 mcg, 200 mcg and 2 mg of SSADH dsRNA applied to roots of sweet orange (Citrus sinensis, cv. Madam Vinous) seedlings grown in growth chamber and moved to the greenhouse.

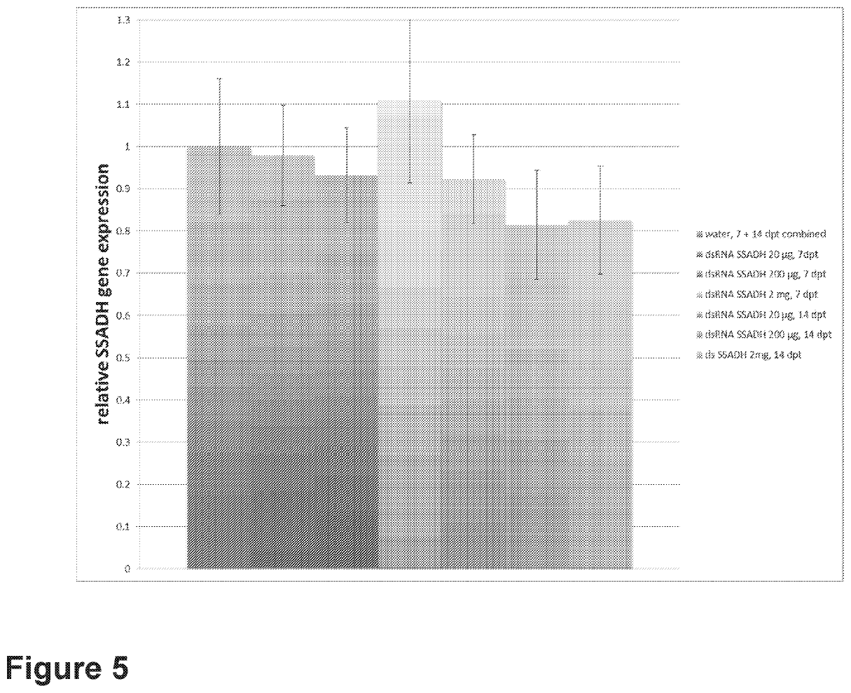

### DETAILED DESCRIPTION OF THE INVENTION

ROS can Trigger Apoptosis

Type I programmed cell death (PCD) or apoptosis, is a genetically programmed and highly regulated cell death mechanism found in plant and animal cells that allows damaged cells to commit suicide. Apoptosis is critically important for elimination of damaged or infected cells that could compromise the function of the whole organism. Typical triggers of apoptosis are environmental insults or stresses that can damage cells or their DNA content. Reactive oxygen species (ROS), including superoxide anions, hydrogen peroxide, nitric oxide and free hydroxide radicals are produced in response to stress, and particularly stress causing mitochondrial damage (Portt, et al., 2011). ROS production per se is a first line of defense in animal and plant cells against biotic disease agents, such as bacteria and fungi. ROS is also one of the major signals that can trigger apoptosis. In addition to ROS production, stress also activates production of the protein “Bax”, and the sphingolipid “ceremide”, and all three are direct proapoptotic messengers. These three major proapoptic messengers can to act independently of one another, since increases in the levels of any one of them (Bax, ROS, or ceramide) is sufficient to trigger apoptosis, but most often, they appear to act in concert. Pathogens that benefit from plant cell death, such as Phytopthora, Ralstonia, Pseudomonas and Xyella are necrotrophic in lifestyle; that is, they kill host cells in order to provide nutrients to sustain in planta population growth. Such pathogens may do little to suppress apoptosis (type I) or necrotic (type III) programmed cell death (Portt, et al., 2011). Other pathogens, such as the obligate fungal parasites (rusts and mildews) and some bacteria, such as Rhizobium, are biotrophic, and must establish intimate cell membrane to membrane contact using haustoria or infection threads.

Programmed Cell Death for Controlling Infections

Liberibacters are the ultimate form of biotroph, living entirely within the living host cell and surrounded by host cell cytoplasm. For biotrophs, host cell death would be expected to severely limit growth in planta. Liberibacters, which must live entirely within living phloem cells, would have no options if their phloem host cells simply died. Biotrophic pathogens typically have multiple mechanisms available to suppress apoptosis or necrotic programmed cell death. In the case of necrotrophic pathogens (those that rely on killing plant cells in order to feed on the contents), suppressing host cell death results in denial of nutrients, and resistance is the result. Necrotrophic pathogens naturally trigger PCD.

Candidatus Liberibacter is a genus of Gram-negative bacteria in the Rhizobiaceae family. Detection of Liberibacters is based on PCR amplification of their 16S rRNA gene with specific primers. Members of the genus are plant pathogens mostly transmitted by psyllids. The genus was originally spelled Liberobacter.

Liberibacters and Other Biotrophic Bacteria

Prototrophic pathogens rely on fully functional living cells to survive. For example, all species and strains of the genus Liberibacter live in plants entirely within living plant phloem cells. The first Liberibacter species described was Candidatus Liberibacter asiaticus (Las), the causal agent of Huanglongbing (HLB), commonly known as citrus “greening” disease. HLB is lethal to citrus and is one of the top three most damaging diseases of citrus. The second species described was found in Africa, Ca. L. africanus (Laf), and the third, Ca. Liberibacter americanus (Lam) was found in Brazil. All three cause HLB in citrus. Beside the three citrus Liberibacters associated with HLB, three non-citrus Liberibacter species have been described. Ca. L. solanacearum (Lso), has been identified as the causal agent of serious diseases of potato (“Zebra chip”), tomato (“psyllid yellows”) and other solanaceous crops in the USA, Mexico, Guatemala, Honduras, and New Zealand (Hansen, et al., 2008; Abad, et al., 2009; Liefting, et al., 2009; Secor, et al., 2009). More recently, a different haplotype of Lso was found infecting carrots in Sweden, Norway, Finland, Spain and the Canary Islands (Alfaro-Femandez, et al., 2012a, 2012b Munyaneza, et al., 2012a, 2012b; Nelson, et al., 2011). A fifth species of Liberibacter, Ca. L. europaeus (Leu) was recently found in the psyllid Cacopsylla pyri, the vector of pear decline phytoplasma. Finally, a sixth species of Liberibacter, Liberibacter crescens (Lcr). was characterized after isolation from diseased mountain papaya (Babaco). Except for Lcr, which is not known to be pathogenic, all other described Liberibacters are pathogenic and must be injected into living plant cells by specific insects. Furthermore, the pathogenic Liberibacters can only live within specific insect and plant cells; as obligate parasites, they do not have a free-living state—they are extreme biotrophs.

As an example of an ordinary biotroph, Xanthomonas citri, which causes citrus canker disease, invades the air spaces within a leaf and relies on inducing cell divisions in living cells in order to rupture the leaf surface (Brunings and Gabriel, 2003). Obviously, for biotrophs, host cell death would be expected to severely limit growth in planta. Liberibacters, which must live entirely within living phloem cells, would have no options if their phloem host cells simply died.

Liberibacter Mechanisms of Avoiding or Suppressing ROS and Programmed Cell Death

The genomes of Las and Lam differ (among other things) in that most Las strains have 4 copies of peroxidase (Zhang, et al., 2011), and most Lam strains have 2 copies (Wulff, et al., 2014). These are critical lysogenic conversion genes (conferring ability to colonize a plant or insect). With both Las and Lam (and likely Lso), these genes are amplified in copy number on a plasmid prophage to increase transcript copy number, and therefore, protein levels (Zhang, et al., 2011). Peroxidases degrade reactive oxygen species (ROS), like hydrogen peroxide. ROS production is one of the primary insect and plant host defenses against microbes. Since Liberibacters colonize living phloem cells and multiply within the plant cell cytoplasm, the ability to degrade ROS is a critical matter of survival to Liberibacter. In addition to the peroxidase genes, all pathogenic Liberibacters encode two peroxiredoxins on their chromosomes. One of the peroxiredoxins is secreted, and not only degrades ROS, but also travels outside the bacteria to prevent peroxidative degradation of lipid membranes in planta, thereby preventing a chain reaction peroxidation event and subsequent accumulation of antimicrobial oxylipins, that are not only antimicrobial but which can also trigger PCD (Jain et al 2018).

Both peroxiredoxins and peroxidases actively suppress PCD, and since ROSs are strong pro-apoptotic inducers of PCD, particularly under certain nutrient deficiencies, the ability to absorb and degrade ROS is a matter of survival for bacteria that need to keep their host cells alive. Since Liberibacters can occupy a significant volume of host cell cytoplasm, the ability to absorb and degrade ROS appears to be critical to suppressing citrus cell apoptosis. Lam causes a lot more disease than Las, but is always present in significantly lower titer (Lopes, et al., 2009). It is possible that the higher titer of Las is allowed by the activity of four peroxidases and two peroxiredoxins, which in combination with the higher titer allow a greater total absorption and inactivation of ROS, reducing the ROS induced proapoptotic effect on the citrus cell.

Plant pathogens provide a series of molecular signals that are detected by the plant and can trigger PCD. These signals, or “pathogen associated molecular patterns” (PAMPs) are detected by plants as alien molecules and trigger strong defense responses called “innate immunity” in plants. Avoidance of triggering PCD by biotrophs involves eliminating by evolution over time, to the greatest possible extent, production of PAMPs.

The genome sequences of both Las and Lam are highly reduced in size (both are 1.26 Mb) as compared with their closest Rhizobium relatives (genome sizes >6.4 Mb); significantly, both Las and Lam appear to lack flagella, a known PAMP, although both encode structural genes for flagellin. In addition, Lam lacks most of the genes needed to make lipopolysaccharide (LPS), a particularly potent PAMP and an important defensive barrier molecule integral to the outer membrane of most Gram negative bacteria.

Plant Regulators of ROS production.

Spatiotemporal regulation of ROS generation and detoxification pathways is critical in plants for maintaining redox poise while preventing oxidation of cellular macromolecules (Schieber and Chandel 2014). Failure to maintain redox poise triggers PCD. Oxidative damage is strongly exacerbated in cells by the production of gamma hydroxybutyrate (GHB) (Gupta et al. 2003), an enzyme located in the mitochondria. The activity of GHB, along with other mitochondrial enzymes that comprise the gamma aminobutyrate (GABA) shunt, is drastically enhanced in plants by both biotic and abiotic stresses, (Bouche et al, 2003, and references therein). One of these key mitochondrial enzymes of the GABA shunt is succinic semialdehyde dehydrogenase (SSADH), which converts succinic semialdehyde to succinate. In competition with the enzyme SSADH, succinic semialdehyde also gets converted to GHB by the action of succinic semialdehyde reductatse (Fait et al., 2005). In Arabidopsis, an SSADH knockout mutation resulted in overproduction of GHB, and consequently overproduction of ROS, necrotic lesions, dwarfism, and hypersensitivity to environmental stress (Fait et al. 2005). This invention is based in part on the inventors' discovery that moderate and transient down regulation of expression of the citrus SSADH gene through the use of siRNA resulted in resistance to a biotrophic plant pathogen, without necrosis or dwarfism in the treated plants that would be expected from a fully mutated or nonfunctional SSADH gene.

In some embodiments, the present invention teaches that the down-regulation of SSADH in plants leads to increased plant resistance to pathogens. In some embodiments, the present disclosure teaches the down-regulation of the citrus SSADH gene leads to increased resistance to biotrophic pathogens. In other embodiments, the disclosure teaches down-regulation of the SSADH gene leads to increased resistance to citrus greening disease caused by Las, Lam, and Laf. By extension, the disclosure teaches down-regulation of the SSADH gene of any specific crop plant leads to increased resistance to biotrophic pathogens, and specifically, Liberibacters that may infect them.

In some embodiments, the present disclosure teaches the down-regulation of t h e sweet orange SSADH gene (SEQ ID NO: 1). In some embodiments, the present invention teaches adequate methods to achieve the transient and effective down regulation of the citrus SSADH in any citrus variety. In some embodiments, the present invention teaches the transient and effective down regulation of the SSADH enzyme in any plant species.

It has not been anticipated, expected, nor suggested in any plant system that transient silencing of an SSADH gene, which results in suppressing production of the corresponding SSADH enzyme, could increase levels of expression of downstream defense response proteins in response to pathogen presence and be protective against pathogen infection or the effects of pathogen infections.

The disclosure here that silencing of SSADH in citrus enhances production of defense responses and is protective from the effects of biotrophic pathogen infection is therefore a surprising discovery.

For gene silencing purposes, both antisense RNA or RNAi may be used instead to activate the RNAi pathway, although the processes result in differing magnitudes of the same downstream effects.

Other Plant Regulators of Programmed Cell Death

Anti-apoptosis proteins have distinct and often specific anti-apoptotic effects when expressed in transgenic plants. Overexpression of many of the genes encoding these anti-apoptotic factors, including genes encoding a variety of chaperones, heat shock proteins and ROS scavengers, such as superoxide dismutase and peroxidases, can prevent apoptosis and are therefore cytoprotective from the effects of biotic and abiotic stresses.

The Bcl-2 family is a well-characterized group of “core” anti-apoptotic regulators with strong general effects; these proteins inhibit production of Bax, and without affecting production of ROS. The Bcl-2 proteins, if overexpressed, broadly inhibit apoptosis in plants caused by both biotic and abiotic stresses. For example, expression of antiapoptotic genes bcl-xL (derived from chickens) and ced-9 (derived from Caenorhabditis elegans) blocked apoptosis in tomato and enhanced tolerance to viral induced necrosis and abiotic stress (Xu, et al., 2004). Similarly, expression of antiapoptotic genes bcl-2 (derived from humans), and op-iap (derived from baculovirus) blocked apoptosis in tobacco and conferred resistance to several necrotrophic fungal pathogens (Dickman, et al., 2001). No plant homologs of Bcl-2 have been identified to date.

There are also reports of an anti-apoptotic role for basic, but not acidic, pathogenesis related (PR) proteins, and many of these affect both biotic and abiotic stress induced responses. For example, a basic PR-1 protein from pepper, when overexpressed in transgenic tobacco, was shown to enhance heavy metal tolerance and to provide significantly higher resistance against Phytopthora, Ralstonia solanacearum and Pseudomonas syringae (Sarowar, et al., 2005). In all cases, necrosis in the abiotically- or biotically-stressed transgenic lines was greatly reduced compared to control lines. Clearly, the necrosis elicited by these pathogens is not a direct effect of pathogenic enzymes, but an indirect effect that triggers PCD that results in necrosis. Similarly, a basic PR-1 protein from grape, when overexpressed in transgenic tobacco, was shown to provide significantly reduced disease lesions in challenge inoculations with P. syringae (Zhijian, et al., 2010). Gilchrist and Lincoln (2011) reported that a grape PR-1 protein, when expressed in several transgenic host species, would suppress apoptosis, and in so doing, provide greatly improved tolerance to Xylella fastidiosa by blocking symptom induction, which in turn leads to a major reduction in total bacterial growth in planta. It should be noted that only a few members from the plant PR-1 family showed inducible expression by pathogens and possessed inhibitory activity against pathogens (Li, et al., 2011). For example, Arabidopsis and rice contain 22 and 39 PR-1 type genes, but only 1 and 2 members, respectively, have been found to be inducible by pathogen or insect attacks (Van Loon, et al., 2006).

BAG6 as a Regulator of Programmed Cell Death

The down regulation of the BAG6 gene in plants leads to an increased programmed cell death response to pathogen incursions. The down-regulation of BAG6 in plants also leads to increased production of defense response protein NDR-1. This increased NDR-1 production in BAG6 silenced plants is particularly significant, since elevated levels of this gene are known to increase basal resistance to both bacteria and fungi in Arabidopsis, and is suggested to be likely to increase resistance to Las in citrus (Lu, et al., 2013).

The SSADH effect on ROS production is thought to be mediated by gamma-hydroxybutyric acid (GHB) which accumulates when there is SSADH deficiency (Fait et al. 2005, FEBS Letters 579:415-420), and also is associated with generation of very high levels of ROS production. This effect is completely independent of cytoprotective role of BAG6 in the chain of events precipitating programmed cell death responses.

The inventors of the present application found the unexpected and surprising result that down-regulation of SSADH can increase plant resistance to pathogens significantly. Thus in some embodiments, the present disclosure teaches the down-regulation of SSADH genes for increasing plant resistance to pathogens. In some embodiments, the present disclosure teaches the down-regulation of SSADH genes to increase resistance of biotrophic pathogens. In other embodiments, the disclosure teaches down-regulation of SSADH genes to increase resistance to citrus greening disease caused by Las, Lam, and Lso.

In some embodiments, the present disclosure teaches the down-regulation of sweet orange SSADH (SEQ ID NO: 1). In some embodiments, the present disclosure teaches the down-regulation of Hamlin-SSADH. In some embodiments, the present disclosure teaches the down-regulation of Valencia-SSADH. In some embodiments, the present disclosure teaches the down-regulation of grapefruit-SSADH.

It has not been anticipated, expected, nor suggested in any plant system that silencing of a SSADH gene, which results in suppressing production of the corresponding SSADH protein, could increase levels of expression of downstream defense response proteins and be protective against pathogen infection or the effects of pathogen infections. The disclosure here that silencing of SSADH in citrus enhances production of defense response proteins and is protective from the effects of pathogen infection is therefore a surprising discovery.

For gene silencing purposes, both antisense RNA or siRNA may be used instead activate the RNAi pathway, although the processes result in differing magnitudes of the same downstream effects.

Controlled Regulation of SSADH by Selective Spatial Temporal Silencing

By artificially increasing the ROS response and precipitating programmed cell death (PCD), specifically sensitizing the plants to detect the presence of a pathogen, the present disclosure teaches effective defenses in response to pathogens in an unusually rapid manner. If this increase in ROS occurred generally in all plant tissues, it could have the undesirable result of making the plant more susceptible to necrotrophic pathogens.

However, if the increase in ROS can be limited to just the phloem and nearby cells, then there should be no necrotrophic pathogen advantage, since necrotrophs do not attack phloem as a first target. Since Liberibacter and Xanthomonas are two bacterial genera that cause major plant diseases and are prototrophic, selective increase in ROS production can confer immunity to Liberibacter, Xanthomonas and other biotrophic pathogens, including fungi, without undesired effects of increasing susceptibility to necrotrophs.

In some embodiments, the present invention provides the use of two RNA-based methods for selectively silencing citrus genes in such a way that: 1) only the rootstock is transgenic, leaving the scion grafted onto the rootstock nontransgenic; 2) the RNA used is limited to phloem cells only (where Las resides), and 3) the RNA used in another case allows limited migration to cells outside the phloem. The present invention provides compositions and methods for effectively increasing the basal defense responses and disease resistance in a plant cell, a plant part, a plant, or a plant population, such as nontransgenic citrus scions or nontransgenic, infected citrus. This disclosure is of significant benefit to U.S. fruit crop producers or producers around the world, since the resulting plants became resistant to severe disease

Sequence Identity

In some embodiments, the present invention teaches the down-regulation of a SSADH homolog or ortholog, in which said homolog or ortholog shares at least 99.9%, 99.8%, 99.7%, 99.6%, 99.5%, 99.4%, 99.3%, 99.2%, 99.1%, 99%, 98.9%, 98.8%, 98.7%, 98.6%, 98.5%, 98.4%, 98.3%, 98.2%, 98.1%, 98%, 97.9%, 97.8%, 97.7%, 97.6%, 97.5%, 97.4%, 97.3%, 97.2%, 97.1%, 97%, 96.9%, 96.8%, 96.7%, 96.6%, 96.5%, 96.4%, 96.3%, 96.2%, 96.1%, 96%, 95.9%, 95.8%, 95.7%, 95.6%, 95.5%, 95.4%, 95.3%, 95.2%, 95.1%, 95%, 94.9%, 94.8%, 94.7%, 94.6%, 94.5%, 94.4%, 94.3%, 94.2%, 94.1%, 94%, 93.9%, 93.8%, 93.7%, 93.6%, 93.5%, 93.4%, 93.3%, 93.2%, 93.1%, 93%, 92.9%, 92.8%, 92.7%, 92.6%, 92.5%, 92.4%, 92.3%, 92.2%, 92.1%, 92%, 91.9%, 91.8%, 91.7%, 91.6%, 91.5%, 91.4%, 91.3%, 91.2%, 91.1%, 91%, 90.9%, 90.8%, 90.7%, 90.6%, 90.5%, 90.4%, 90.3%, 90.2%, 90.1%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, or 40% sequence identity with SEQ ID NO: 1, SEQ ID NO: 3, SEQ ID NO: 6, SEQ ID NO: 7 or SEQ ID NO: 8.

In some embodiments, the present invention the down-regulation of a SSADH homolog or ortholog, in which said homolog or ortholog shares nucleotide sequence encoding an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%, sequence identity to SEQ ID NO: 2 or SEQ ID NO: 4. In some embodiments, the sequence identity of the SSADH homolog or ortholog is calculated based on the alignment and comparison of each gene's nucleic acid sequence. In other embodiments, the sequence identity of the homolog or ortholog is calculated based on the alignment and comparison of the encoded protein.

In some embodiments, the alignments and sequence identity calculations of the present invention are calculated using ClustalOmega software with default settings.

In some embodiments, the SSADH gene homologs and orthologs of the present disclosure will encode for conservative amino acid substitutions. Conservative amino acid substitutions are those substitutions that, when made, least interfere with the properties of the original protein, that is, the structure and especially the function of the protein is conserved and not significantly changed by such substitutions. Conservative substitutions generally maintain: (a) the structure of the polypeptide backbone in the area of the substitution, for example, as a sheet or helical conformation; (b) the charge or hydrophobicity of the molecule at the target site; or (c) the bulk of the side chain. Further information about conservative substitutions can be found, for instance, in Ben Bassat, et al. (J. Bacteriol., 169:751-757, 1987), O'Regan, et al. (Gene, 77:237-251, 1989), Sahin-Toth, et al. (Protein Sci., 3:240-247, 1994), Hochuli, et al. (Bio/Technologv, 6:1321-1325, 1988) and in widely used textbooks of genetics and molecular biology. The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff, et al., Proc. Natl. Acad Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. The following table shows exemplary conservative amino acid substitutions.

TABLE 1
BLOSUM substitution Matrix

| | | Highly | |
|---|---|---|---|
| | | Conserved | Conserved |
| | Very | Substitutions | Substitutions |
| | Highly - | (from the | (from the |
| Original | Conserved | Blosum90 | Blosum65 |
| Residue | Substitutions | Matrix) | Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, | Arg, Asp, Gln, Glu, His, |
| | | Lys, Ser, Thr | Lys, Ser, Thr |
| Asp | Glu | Asn, Gln | Asn, Gln, Glu, Ser |
| Cys | Set | None | Ala |
| Gln | Asn | Arg, Asn, Glu, | Arg, Asn, Asp, Glu, His, |
| | | His, Lys, Met | Lys, Met, Ser |
| Gln | Asp | Asp, Gln, Lys | Arg, Asn, Asp, Gln, His, |
| | | | Lys, Ser |
| Gly | Pro | Ala | Ala, Ser |
| His | Asn, Gln | Arg, Asn, Gln, | Arg, Asn, Gln, Glu, Tyr |
| | | Tyr | |
| Ile | Leu; Val | Leu, Met, Val | Leu, Met, Phe, Val |
| Leu | Ile; Val | Ile, Met, Phe, | Ile, Met, Phe, Val |
| | | Val | |
| Lys | Arg; Gln; Glu | Arg, Asn, Gln, | Arg, Asn, Gln, Glu, Ser, |
| | | Glu | |
| Met | Leu; Ile | Gln, Ile, Leu, | Gln, Ile, Leu, Phe, Val |
| | | Val | |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, |
| | | | Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

In some embodiments, orthologs and homologs of the present invention can have no more than 3, 5, 10, 15, 20, 25, 30, 40, 50, or 100 conservative amino acid changes (such as very highly conserved or highly conserved amino acid substitutions). In other examples, one or several hydrophobic residues (such as Leu, Ile, Val, Met, Phe, or Trp) in a variant sequence can are found to be replaced with a different hydrophobic residue (such as Leu, Ile, Val, Met, Phe, or Trp) to create a variant functionally similar to the disclosed an amino acid sequences encoded by the nucleic acid sequences of SSADH.

In some embodiments, variants may differ from the disclosed sequences by alteration of the coding region to fit the codon usage bias of the particular organism into which the molecule is to be introduced. In other embodiments, the coding region may be altered by taking advantage of the degeneracy of the genetic code to alter the coding sequence such that, while the nucleotide sequence is substantially altered, it nevertheless encodes a protein having an amino acid sequence substantially similar to the disclosed an amino acid sequences encoded by the nucleic acid sequences of SSADH.

Recombinant DNA Constructs

Another aspect of this invention provides a recombinant nucleic acid construct including a heterologous promoter operably linked to DNA including at least one segment of 15, 16, 17, 18, 19, 20, 21, 22, 23, 24, or 25 more contiguous nucleotides with a sequence of about 70% to about 100% identity with a segment of equivalent length of a DNA having a sequence selected from the group consisting of SEQ ID NO:1, SEQ ID NO:3, SEQ ID NO: 6; SEQ ID NO: 7, and SEQ ID NO: 8. The recombinant nucleic acid constructs are useful in providing a plant having improved resistance to bacterial or fungal infections, e.g., by expressing in a plant a transcript of such a recombinant nucleic acid construct. The contiguous nucleotides can number more than 15, 16, 17, 18, 19, 20, 21, 22, 23, 24, 25, 26, 27, 28, 29, 30, or greater than 30, e.g., about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, or greater than 900 contiguous nucleotides from SEQ ID NO:1 or SEQ ID NO:3, SEQ ID NO: 6; SEQ ID NO: 7, or SEQ ID NO: 8.

The contiguous nucleotides can number more than about 900, about 1000, about 1100, about 1200, about 1300, about 1400, about 1500, about 1600, about 1700, about 1800, about 1900, about 2000, about 2100, about 2200, about 2300, about 2400, about 2500 contiguous nucleotides from SEQ ID NO:1, SEQ ID NO:3, SEQ ID NO: 6; SEQ ID NO: 7, or SEQ ID NO: 8.

In some embodiments, the recombinant nucleic acid construct of this invention is provided in a recombinant vector. By “recombinant vector” is meant a recombinant polynucleotide molecule that is used to transfer genetic information from one cell to another. Embodiments suitable to this invention include, but are not limited to, recombinant plasmids, recombinant cosmids, artificial chromosomes, and recombinant viral vectors such as recombinant plant virus vectors and recombinant baculovirus vectors.

RNA Interference

Sequence-selective, post-transcriptional inactivation of expression of a target gene can be achieved in a wide variety of eukaryotes by introducing double-stranded RNA (dsRNA) corresponding to the target gene, a phenomenon termed RNA interference (RNAi). RNAi occurs when an organism recognizes dsRNA molecules and hydrolyzes them. The resulting hydrolysis products are small RNA fragments of 15, 16, 17, 18, 19, 20, 21, 22, 23 or 24 nucleotides in length, called small interfering RNAs (siRNAs) or microRNAs (miRNAs). The siRNAs then diffuse or are carried throughout the organism, including across cellular membranes, where they hybridize to mRNAs (or other RNAs) and cause hydrolysis of the RNA. Most plant miRNAs show extensive base pairing to, and guide cleavage of their target mRNAs (Jones-Rhoades et al. (2006) Annu. Rev. Plant Biol. 57, 19-53; Llave et al. (2002) Proc. Natl. Acad. Sci. USA 97, 13401-10406). In other instances, interfering RNAs may bind to target RNA molecules having imperfect complementarity, causing translational repression without mRNA degradation.

The term “RNAi” or “RNA interference” refers to the process of sequence-specific post-transcriptional gene silencing (e.g., in nematodes), mediated by double-stranded RNA (dsRNA). “DsRNA” refers to RNA that is partially or completely double stranded. Double stranded RNA is also referred to as small interfering RNA (siRNA), small interfering nucleic acid (siNA), microRNA (miRNA), and the like. In the RNAi process, dsRNA comprising a first (antisense) strand that is complementary to a portion of a target gene and a second (sense) strand that is fully or partially complementary to the first antisense strand is introduced into an organism (e.g., plants and/or crops), by, e.g., transformation, injection, spray, brush, mechanical abrasion, laser etching or immersion, etc. After introduction into the organism, the target gene-specific dsRNA is processed into relatively small fragments (siRNAs) and can subsequently become distributed throughout the organism, leading to a loss-of-function mutation having a phenotype that, over the period of a generation, may come to closely resemble the phenotype arising from a complete or partial deletion of the target gene.

This approach takes advantage of the discovery that siRNA can trigger the degradation of mRNA corresponding to the siRNA sequence. RNAi is a remarkably efficient process whereby dsRNA induces the sequence-specific degradation of homologous mRNA in animals and plant cells (Hutvagner and Zamore (2002), Curr. Opin. Genet. Dev., 12, 225-232; Sharp (2001), Genes Dev., 15, 485-490).

The effects of RNAi can be both systemic and heritable in plants. In plants, RNAi is thought to propagate by the transfer of siRNAs between cells through plasmodesmata. The heritability comes from methylation of promoters targeted by RNAi; the new methylation pattern is copied in each new generation of the cell. A broad general distinction between plants and animals lies in the targeting of endogenously produced miRNAs; in plants, miRNAs are usually perfectly or nearly perfectly complementary to their target genes and induce direct mRNA cleavage by RISC, while animals' miRNAs tend to be more divergent in sequence and induce translational repression. Detailed methods for RNAi in plants are described in David Allis et al (Epigenetics, CSHL Press, 2007, ISBN 0879697245, 978087%97242), Sohail et al (Gene silencing by RNA interference: technology and application, CRC Press, 2005, ISBN 0849321417, 9780849321412). Engelke et al. (RAN Interference, Academic Press, 2005, ISBN 0121827976, 9780121827977), and Doran et al. (RNA Interference: Methods for Plants and Animals, CABI, 2009, ISBN 1845934105, 9781845934101), which are all herein incorporated by reference in their entireties for all purposes.

The term “dsRNA” or “dsRNA molecule” or “double-strand RNA effector molecule” refers to an at least partially double-strand ribonucleic acid molecule containing a region of at least about 19 or more nucleotides that are in a double-strand conformation. The double-stranded RNA effector molecule may be a duplex double-stranded RNA formed from two separate RNA strands or it may be a single RNA strand with regions of self-complementarity capable of assuming an at least partially double-stranded hairpin conformation (i.e., a hairpin dsRNA or stem-loop dsRNA). In various embodiments, the dsRNA consists entirely of ribonucleotides or consists of a mixture of ribonucleotides and deoxynucleotides, such as RNA/DNA hybrids. The dsRNA may be a single molecule with regions of self-complementarity such that nucleotides in one segment of the molecule base pair with nucleotides in another segment of the molecule.

DsRNA-mediated regulation of gene expression in plants is well known to those skilled in the art. See, e.g., WIPO Patent Application Nos. WO1999/061631A and WO1999/053050A, each of which is incorporated by reference herein in its entirety.

As used herein, “RNA transcript” refers to the product resulting from RNA polymerase-catalyzed transcription of a DNA sequence. When the RNA transcript is a perfect complementary copy of the DNA sequence, it is referred to as the primary transcript. An RNA transcript is referred to as the mature RNA when it is an RNA sequence derived from post-transcriptional processing of the primary transcript. “Messenger RNA (mRNA)” refers to the RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. The terms “complementary” and “reverse complementary” are used interchangeably herein with respect to mRNA transcripts, and are meant to define the antisense RNA of the message.

In some embodiments, an RNAi agent includes a single stranded RNA that interacts with a target RNA sequence to direct the cleavage of the target RNA. Without wishing to be bound by theory, long double stranded RNA introduced into plants and invertebrate cells is broken down into siRNA by a Type III endonuclease known as Dicer (Sharp et al., Genes Dev. 2001, 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev. 15:188).

In some embodiments the present invention also teaches expression vectors capable of producing inhibitor nucleic acid molecules. In some embodiments, the present invention teaches the use of RNA interference (RNAi) for the down-regulation of SSADH genes, or homologs or orthologs of SSADH genes. Thus in some embodiments the present invention teaches the expression of antisense, inverted repeat, small RNAs, artificial miRNA, or other RNAi triggering sequences.

In some embodiments the RNAi constructs of the present invention comprise sequences capable of triggering RNAi suppression of SSADH genes, including nucleic acid fragments comprising sequence identities higher than about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% to a SSADH gene target region, such as those disclosed in SEQ ID NO: 1, SEQ ID NO:3, SEQ ID NO: 6; SEQ ID NO: 7, or SEQ ID NO: 8.

In some embodiments, the antisense or small RNA molecules are targeted to a section of the coding portion of the target gene. In other embodiments, the RNAi sequences of the present invention are targeted to the 5′ or 3′ untranslated regions (UTRs) of the target gene. In yet other embodiments, the RNAi sequences of the present invention are targeted to the promoter of the target gene. Methods of selecting sequence target regions for RNAi molecule design are described in more detail in (Fougerolles, et al., 2007; U.S. Pat. No. 7,732,593).

In some embodiments, the RNAi molecules of the invention may be modified at various locations, including the sugar moiety, the phosphodiester linkage, and/or the base. For example, in order to further increase the stability of the molecules in vivo, the 3′-end of the hairpin structure may be blocked by protective group(s). For example, protective groups such as inverted nucleotides, inverted abasic moieties, or amino-end modified nucleotides may be used. Inverted nucleotides may comprise an inverted deoxynucleotide. Inverted abasic moieties may comprise an inverted deoxyabasic moiety, such as a 3′,3′-linked or 5′,5′-linked deoxyabasic moiety (U.S. Patent Publication 2011/251258).

In some embodiments, the present invention also teaches the down-regulation of genes via antisense technology. In some embodiments, the present invention can be practiced using other known methods for down-regulating gene expression including T-DNA knockout lines, tilling, TAL-mediated gene disruption, transcriptional gene silencing, and site-directed methylations.

Application of RNAi Formulation/Treatment

Plant recombinant technology is the vehicle for delivering gene silencing of target genes, either endogenous plant target genes or target genes of a plant pest organism. In general, a plant is transformed with DNA that is incorporated into the plant genome, and when expressed produces a dsRNA that is complementary to a gene of interest, which can be an endogenous plant gene or an essential gene of a plant pest. Plant recombination techniques to generate transgene and beneficial plant traits require significant investments in research and development, and pose significant regulatory hurdles. Methods and formulations for delivering dsRNA into plant cells by exogenous application to exterior portions of the plant, such as leaf, stem, and/or root surfaces for regulation of endogenous gene expression are known in the art. See, e.g., U.S. Pat. No. 9,433,217, U.S. Patent Publication 2013/0047298, Chinese Patent No. 103748230B and Chinese Patent Publication CN101914540A, each of which is incorporated by reference herein in its entirety. Such methods and formulations represent a significant development for gene silencing technology using RNAi.

In some embodiments, the present invention teaches methods and formulations to topically apply exogenous RNA molecules to external tissue surfaces of plants. In some embodiments, the application exogenous RNA molecules, including dsRNA, siRNA, miRNA and aRNA, causes silencing of plant endogenous target genes or of the target genes of plant pests in the plant cells nearby the external tissue surfaces. In some embodiments, the application exogenous RNA molecules, including dsRNA, siRNA, miRNA and aRNA, causes silencing of plant endogenous target genes or of the target genes of plant pests in the plant cells that is located in a long distance from the external tissue surfaces.

In some embodiments, the present invention provides that applying dsRNA formulations (and/or treatments) by spray, brush, mechanical abrasion, laser etching or immersion of the dsRNA molecules, or other non-tissue invasive techniques, leads to absorption and assimilation of the exogenous RNA molecules into nearby or distant plant cells, thus causing endogenous and/or pest gene silencing. In some embodiments, pest genes are introduced into host plants by bacterial, fungal, or viral infection.

In some embodiments, the present invention teaches methods of repressing, preventing, eliminating, reducing, or otherwise ameliorating a bacterial or fungal infection of a plant comprising topical application of nucleic acid including DNA molecules as well as RNA molecules including dsRNA, siRNA, miRNA and aRNA.

The following examples illustrate various aspects of the invention. The examples should, of course, be understood to be merely illustrative of only certain embodiments of the invention and not to constitute limitations upon the scope of the invention.

### Example 1: Identification of a Citrus SSADH Homolog and Demonstration of Single Identical Homologs in Sweet Orange and Grapefruit

BLAST-P searches were performed using the predicted SSADH protein sequence from Arabidopsis (SEQ ID NO: 4) to identify a single citrus locus within the Valencia sweet orange (Citrus sinensis) cv. Valencia genome available online referred to herein as Valencia SSADH (SEQ ID NO: 2), encoded by the gene region provided in SEQ ID NO: 1. The DNA sequence of this locus, which is an SSADH homolog, was then used to determine how closely related it might be to other members of the SSADH gene family in citrus. No citrus varieties, including C. sinensis (sweet orange) cvs. Valencia. Hamlin and Madam vinous or C. paradisi (grapefruit) appeared to have any other sequences with high sequence similarity (>50%) over a stretch of more than 18 base pairs to SSADH. This demonstrated not only that citrus did not likely carry multiple SSADH homologs, but also that most regions on Valencia SSADH chosen as a probe or for silencing purposes would likely be specific to the target SSADH gene. Citrus mRNA was isolated from four commercially planted citrus species or varieties, including C. sinensis cv. Valencia (sweet orange), C. sinensis cv. Hamlin (sweet orange), C. paradisi (grapefruit), and Citrus sinensis (L.) X Poncirius trifoliata (Carrizo, a rootstock). qRT-PCR was performed on all these mRNAs using standard methods (Jiang, et al., 2012) to amplify a 930 bp exon fragment using appropriate PCR primers. These DNA fragments were cloned into pGEM-T and three clones from each of the three citrus varieties were sequenced. The DNA sequences were 98% identical over the entire 2209 bp stretch of SSADH from Valencia sweet orange (SEQ ID NO: 1), and nearly 100% identical (1 bp difference between Valencia and grapefruit (SEQ ID NO: 6), Hamlin (SEQ ID NO: 7) or Carrizo (SEQ ID NO: 8) Valencia over the first 200 bp of SEQ ID NO: 1. Not surprisingly, there were additional single nucleotide polymorphisms and a 24 bp addition to Carrizo not found in the 5′ untranslated region of the other citrus species. This demonstrated that most regions on Valencia SSADH chosen as a probe or for silencing purposes would likely be specific to the target SSADH gene.

### Example 2. SSADH Expression was Suppressed in Nontransgenic Citrus Sweet Orange Leaves within 3 Days after Placing the Cut Stems of Seedlings into an Aqueous Solution of SSADH dsRNA

In order to determine if exogenously supplied dsRNA could be applied directly to sweet orange citrus in order to silence SSADH, Citrus sinensis cv. Madam vinous seeds were germinated and the seedlings grown hydroponically for 6-8 weeks until the aerial parts of the seedlings were ca. 4″ tall in growth chambers. The roots were removed and immersed in a water solution containing either 5 ng or 100 ng of long dsRNA (500 nt in length) The dsRNA used was commercially prepared to correspond to the DNA coding region of SEQ ID NO. 1 from positions 433-931.

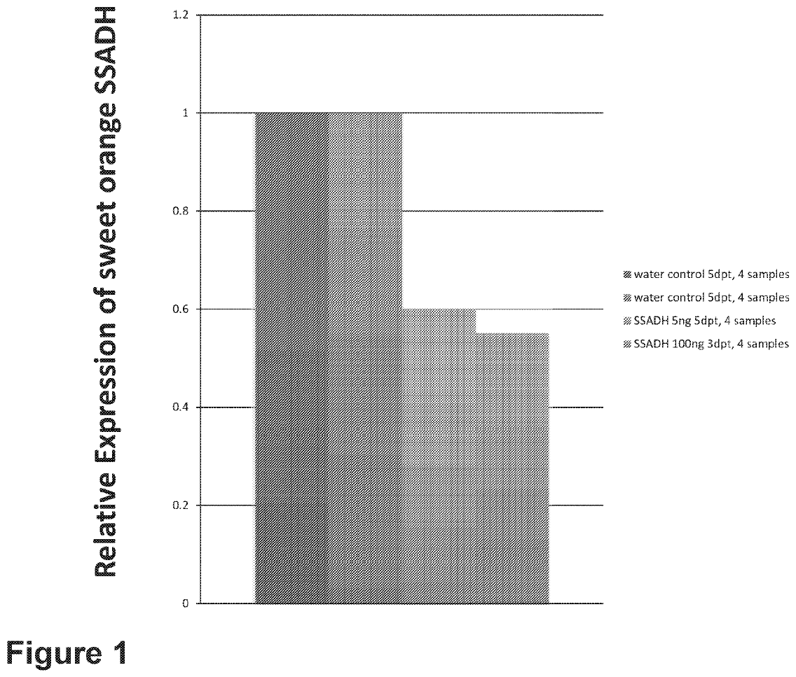

Care was taken to protect the untreated leaves from exposure to the applied dsRNA solution. RNA was extracted from the leaves of the treated plants 3 or 5 days after treatment. Real time quantitative reverse transcription PCR (qRT-PCR) was used to determine levels of expression of SSADH in all samples, using primers well outside of the region of SSADH used for the RNA treatments. qRT-PCR levels were normalized against the control reaction (set at 100%), and relative expression levels of SSADH in both treatments determined. As documented in FIG. 1, dsRNA moved long distances up the stem and into the leaves within 3 days, as evidenced by the suppression of expression of SSADH in the untreated leaves. This demonstrated that 500 nucleotides of citrus SSADH mRNA could be used to silence SSADH in sweet orange citrus within 3 days of treatment at a concentration of 100 ng/4 inch stem (each weighing 150-300 mg) and within 5 days of treatment at 5 ng/4 inch stem.

### Example 3. SSADH dsRNA Formulated as a Nanoemulsion and Used for Foliar Spray Treatments Resulted in Suppression of Ca. Liberibacter asiaticus (Las) Gene Expression in Field Grown Commercial Grapefruit

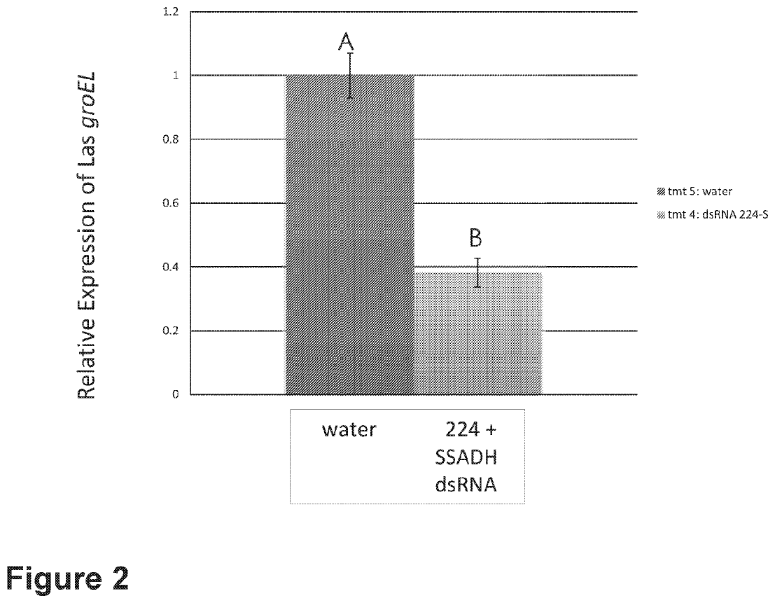

In two separate experimental field trials using five year old grapefruit (Citrus paradisi) trees planted in a commercial grove that was 100% infected with Las, selected grapefruit trees were sprayed with an aqueous nanoemulsion formulation (224) of commercially prepared long (about 500 nt in length) dsRNA and applied at a rate of 0.05 to 0.1 grams per tree. As clearly observed in FIG. 2, Las groEL gene expression, an indicator of living Las cell metabolic activity, was suppressed after single spray treatments at 32 days following treatment. This result demonstrated that spray treatments with citrus SSADH dsRNA formulated as a stabilized nanoemulsions in Formula 224 killed or at least reduced metabolic activity in living Las cells in commercially grown citrus trees in the field. This treatment showed statistically significant effects at by both Student's T and Tuckey's tests at 0.05 confidence levels. Since SSADH is known to suppress ROS generation, this example demonstrates that increased plant capacity to generate ROS is useful in killing or suppressing Las. Statistically significant direct and systemic suppression of metabolic activity of Las live cells in all branches treated with SSADH dsRNA, applied as a simple nano-emulsion spray without laser etching, was observed.

### Example 4. SSADH dsRNA Applied Using Laser Etching and Spray Treatments Resulted in Suppression of Ca. Liberibacter asiaticus (Las) Gene Expression in Field Grown Commercial Grapefruit

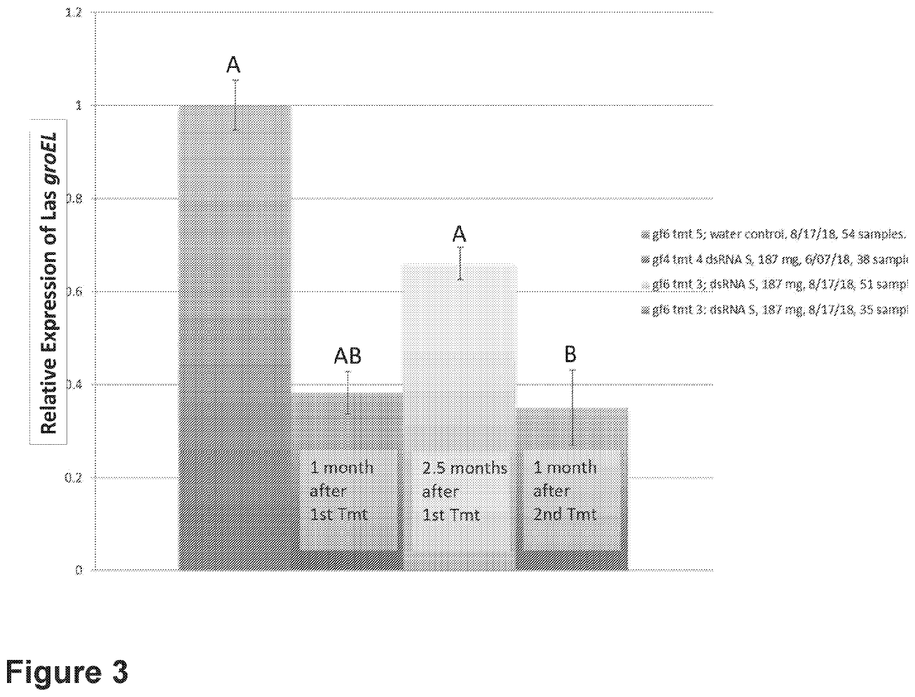

In a separate experimental field trial using five year old grapefruit (Citrus paradisi) trees planted in a commercial grove that was 100% infected with Las, selected grapefruit trees were treated by laser etching to the foliage on one side of the citrus trees using SSADH dsRNA (about 500 nt in length) dsRNA dissolved in water and applied at a rate of 0.187 grams per tree. As clearly observed in FIG. 3, Las groEL gene expression, an indicator of living Las cell metabolic activity, was significantly suppressed after a single spray treatment one month after treatment. The effect wore off after an additional 1.5 months, but retreatment caused an even greater suppressive effect, also significantly different. This result demonstrated that spray treatments with aqueous citrus SSADH dsRNA applied by laser etching killed or at least significantly reduced metabolic activity in living Las cells in commercially grown citrus trees in the field. This treatment showed statistically significant effects at by both Student's T and Tuckey's tests at 0.05 confidence levels. Statistically significant direct and systemic suppression of metabolic activity of Las live cells in all branches, whether directly treated with SSADH dsRNA by laser etching or on untreated leaves on the opposite side of the tree, was observed.

### Example 5. SSADH Alone, or in Combination with BAG6 dsRNA Applied Using Laser Etching and Spray Treatments Resulted in Suppression of Ca. Liberibacter asiaticus (Las) Gene Expression in Field Grown Commercial Grapefruits

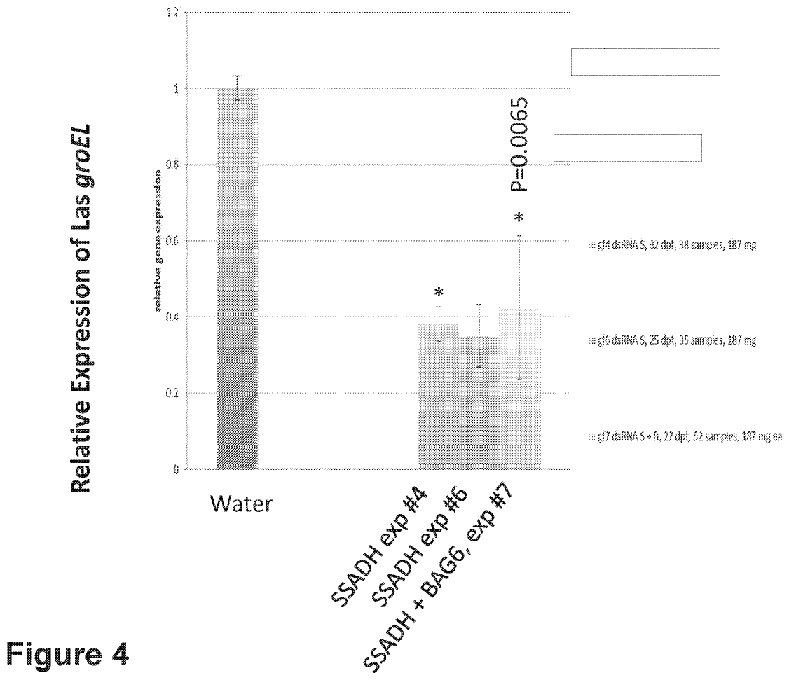

In three separate experimental field trials using five-year-old grapefruits (Citrus paradisi) trees planted in a commercial grove that was 100% infected with Las, selected grapefruit trees were treated by laser etching to the foliage on one side of the citrus trees using SSADH dsRNA or in one experiment to test synergism with an entirely different family of anti-apoptotic genes, with SSADH plus BAG6 dsRNA (both about 500 nt in length) dissolved in water and applied at a rate of 0.187 grams per tree for each dsRNA. As clearly observed in FIG. 4, Las groEL gene expression, an indicator of living Las cell metabolic activity, was significantly suppressed following one SSADH treatment (experiment 4), suppressed but not significantly suppressed following one SSADH treatment (experiment 6) and very significantly suppressed following treatment using a combination of SSADH and BAG6 (experiment 7) after a single spray treatment, observed one month after treatment. This result demonstrated that spray treatments with aqueous citrus SSADH or with SSADH plus BAG6 dsRNA applied by laser etching killed or at least significantly reduced metabolic activity in living Las cells in commercially grown citrus trees in the field. Again, and as in Example 4, direct and systemic suppression of metabolic activity of Las live cells in all branches, whether directly treated with SSADH or SSADH+BAG6 dsRNA by laser etching or on untreated leaves on the opposite side of the tree, was observed. This confirms that dsRNA from both BAG6 (disclosed in WO 2016/086142 A1) and a second, unrelated gene (encoding SSADH) can achieve selective silencing of either SSADH or BAG6 or both using the RNAi application methodologies employed herein or in WO 2016/086142 to increase basal defense responses and disease resistance in nontransgenic infected citrus. The statistical significance of P<0.01 observed using the combination of SSADH and BAG6 was the strongest observed in these experiments, and indicates synergism of suppression of different mechanisms of apoptotic suppression (ROS generation in the case of SSADH and cytoprotective effects in the case of BAG6).

### Example 6. SSADH dsRNA May be Applied by Root Uptake in Citrus to Achieve Gene Silencing of the Target Gene in Sweet Orange

Solutions containing various concentrations of SSADH dsRNA (1×, 10× and 100×) were applied to roots of sweet orange (Citrus sinensis cv. Madam vinous) seedlings grown in a growth chamber and moved to greenhouse. As may be observed in FIG. 6, SSADH applied by root uptake resulted in the suppression of the target SSADH gene in the untreated leaves, this time in a different sweet orange variety. These results demonstrated the validity of using the same sequence of dsRNA to achieve gene silencing on sweet orange varieties Hamlin and Madam vinous, as well as in grapefruits. In addition, taken together with the other examples above, these results expand the potential application methodologies for use of dsRNA to silence SSADH, BAG6 or any other negative regulator of plant defense responses to include nanoemulsions, laser etching, or root uptake.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein.

All publications, patents, patent publications, and nucleic acid and amino acid sequences cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### CITED REFERENCES

de Andrade, E. C., and Hunter, W. B. 2016. RNA Interference-Natural Gene-Based Technology for Highly Specific Pest Control (HiSPeC). http://dx.doi.org/10.5772/61612
Bouché, N., Fait, A., Moller, S. G. and Fromm, H. 2003. Mitochondrial succinic-semialdehyde dehydrogenase of the caminobutyrate shunt is required to restrict levels of reactive oxygen intermediates in plants. Proc. Natl. Acad. Sci. USA 100: 6843-6848.
Brunings, A. M. and Gabriel, D. W. 2003. Xanthomonas citri: breaking the surface. Molecular Plant Pathology 4:141-157.
Chibucos, M. C., C. W. Collmer, T. Torto-Alalibo, M. Gwinn-Giglio, M., Lindeberg, D. Li, and B. M. Tyler. 2009. Programmed cell death in host symbiont associations, viewed through the Gene Ontology. BMC Microbiol. 9 (Suppl. 1): S5.29.
Deng, J.-X., Nie, X.-J., Lei, Y.-F., Ma, C.-F., Xu, D.-L., Li, B. and Zhang, G.-C. 2012. The highly conserved 5′ untranslated region as an effective target towards the inhibition of Enterovirus 71 replication by unmodified and appropriate 2′-modified siRNAs. Journal of Biomedical Science, 19(1), 73. http://doi.org/10.1186/1423-0127-19-73
Dickman, M. B., Park, Y. K., Oltersdorf‡, T., Li, W., Clemente, T., French, R. 2001. Abrogation of disease development in plants expressing animal antiapoptotic genes. Proc. Natl. Acad. Sci. USA 98: 6957-6962.
Doukhanina, E. V., S. Chen, E. van der Zalm, A. Godzik, J. Reed, and M. B. Dickman. 2006. Identification and functional characterization of the BAG protein family in Arabidopsis thaliana. Journal of Biological Chemistry 281:18793-18801
Fait, A., Yellin, A. and Fromm, H. 2005. GABA shunt deficiencies and accumulation of reactive oxygen intermediates, insight from Arabidopsis mutants. FEBS Letters 579:415420
Fittipaldi, M., Nocker, A., Codony, F., 2012. Progress in understanding preferential detection of live cells using viability dyes in combination with DNA amplification.
J. Microbiol. Methods. 91 (2), 276-289.
Gardner, C. L., Pagliai, F. A., Pan, L., Bojilova, L., Torino, M. I., Lorca, G. L., & Gonzalez, C. F. (2016). Drug Repurposing: Tolfenamic Acid Inactivates PrbP, a Transcriptional Accessory Protein in Liberibacter asiaticus. Frontiers in Microbiology, 7, 1630. http://doi.org/10.3389/fmicb.2016.01630.
Gilchrist, D. and Lincoln, J. 2011. Pierce's Disease control and bacterial population dynamics in winegrape varieties grafted to rootstocks expressing anti-apoptotic sequences. Proceedings of the 2011 Pierce's Disease Research Symposium, Sacramento, pp 118-121.
Gupta. M., Hogema, B. M., Grompe, M., Bottiglieri, T. G., Concas, A., Biggio, G., Sogliano, C., Rigamonti, A. E., Pearl, P. L., Snead 3rd, O. C., Jakobs, C. and Gibson, K. M. 2003. Murine succinate semialdehyde dehydrogenase deficiency. Ann. Neurol. 54, S81-S90
Jain, M., Munoz-Bodnar, A., Zhang, S. and Gabriel, D. W., 2018. A Secreted ‘Candidatus Liberibacter asiaticus’ Peroxiredoxin Simultaneously Suppresses Both Localized and Systemic Innate Immune Responses in Planta. Molecular Plant-Microbe Interactions, https://doi.org/10.1094/MPMI-03-18-0068-R.
Jiang, Y., Ding, X., Chen, X., Wang, Y., Song, W. 2013. The rice XA21 binding protein 25 encodes an ankyrin repeat containing protein and is required for full Xa21-mediated disease resistance. Plant J. 73: 814-823.
Kasai A, Bai S, Li T, Harada T (2011) Graft-Transmitted siRNA Signal from the Root Induces Visual Manifestation of Endogenous Post-Transcriptional Gene Silencing in the Scion. PLoS ONE 6(2): e16895.
Lai C F, Chen C Y & Au L C (2013): Comparison between the repression potency of siRNA targeting the coding region and the 3′-untranslated region of mRNA. Biomed Res Int 2013: 637850.
Li Z T, Dhekney S A, Gray D J. 2011. PR-1 gene family of grapevine: a uniquely duplicated PR-1 gene from a Vitis interspecific hybrid confers high-level resistance to bacterial disease in transgenic tobacco. Plant Cell Rep. 30: 1-11.
Lacomme, C. and S. S. Cruz. 1999. Bax-induced cell death in tobacco is similar to the hypersensitive response. Proceedings of the National Academy of Sciences of the United States of America 96, no. 14:7956-7961.
Lopes, S. A., Bertolini, E., Frare, G. F., Martins, E. C., Wulff, N. A., Teixeira, D. C., Femandes, N. G., and Cambra, M. 2009. Graft transmission efficiencies and multiplication of ‘Candidatus Liberibacter americanus’ and ‘Ca. Liberibacter asiaticus’ in citrus plants. Phytopathology 99:301-306
Lu, H., Zhang, C., Albrecht. U., Shimizu, R., Wang, G., Bowman, K. D. 2013. Overexpression of a citrus NDR1 ortholog increases disease resistance in Arabidopsis. Front. Plant Sci. 4: Article 157: 1-10.
Melnyk, C W., Molnar, A. and D. C. Baulcombe 2011. Intercellular and systemic movement of RNA silencing signals. The EMBO Journal 30: 3553-3563.
Portt, L., Norman, G., Clapp, C., Greenwood, M., and Greenwood, M. T. Anti-apoptosis and cell survival: A review. Biochimica et Biophysica Acta 1813: 238-259.
Reynolds, A., D. Leake. Q. Boese, S. Scaringe, W. S. Marshall, and A. Khvorova. 2004. Rational siRNA design for RNA interference. Nature Biotechnology 22:326-330.
Sarowar, S., Y. J. Kim, E. N. Kim, K. D. Kim, B. K. Hwang, R. Islam, and J. S. Shin. 2005. Overexpression of a pepper basic pathogenesis-related protein 1 gene in tobacco plants enhances resistance to heavy metal and pathogen stresses. Plant Cell Reports 24, no. 4:216-224.
Shi, Q., Febres, V J., Jones, J. B., Moore, G. A. 2014. Responsiveness of different citrus genotypes to the Xanthomonas citri ssp.citri-derived pathogen-associated molecular pattern (PAMP) flg22 correlates with resistance to citrus canker. Molecular Plant Pathology. Article first published online; DOI: 10.1111/mpp.12206.
Shi, Q., Febres, V. J., Jones, J. B., Moore, G. A. 2013. Flg22 derived from Xanthomonas citri subsp. citri and ‘Candidatus Liberibacter asiaticus’ trigger similar defense responses in mandarin and grapefruit. Phytopathology. 103(Suppl. 2): S2.132.
Stoutjesdijk P Al, Singh S P, Liu Q. Hurlstone C J, Waterhouse P A, Green A G. 2002. hpRNA-mediated targeting of the Arabidopsis FAD2 gene gives highly efficient and stable silencing. Plant Physiol. 129:1723-31.
Takayama, S., Sato, T., Krajewski, S., Kochel, K., Irie, S., Millan, J. A., and Reed, J. C. 1995. Cloning and functional analysis of BAG-1, a novel BCL-2 binding protein with anti-cell death activity. Cell 80, 279-284).
Van Loon L, Rep M, Pieterse C. 2006. Significance of inducible defense-related proteins in infected plants. Annu. Rev. Phytopathol. 44: 135-162.
Watanabe N. Lam E (2008) BAX inhibitor-1 modulates endoplasmic reticulum stress mediated programmed cell death in Arabidopsis. J Biol Chem 283:3200-3210.
Williams, B., Kabbage, M., Britt, R., and Dickman, M. B. 2010. AtBAG7, an Arabidopsis Bcl-2-associated athano gene, resides in the endoplasmic reticulum and is involved in the unfolded protein response. Proc. Natl. Acad. Sci. USA 107:6088-6093.
Xu, P., Rogers, S. J., Roossinck, M. J., 2004. Expression of antiapoptotic genes bcl-xL and ced-9 in tomato enhances tolerance to viral-induced necrosis and abiotic stress. Proc. Natl. Acad. Sci. USA 101:15805-15810.
Zhang, S., Flores-Cruz, Z., Zhou, L., Kang, B. H., Fleites, L., Gooch, M. D., Wulff. N. A., Davis, M. J., Duan, Y., and Gabriel, D. W. 2011. Ca. Liberibacter asiaticus carries an excision plasmid prophage and a chromosomally integrated prophage that becomes lytic in plant infections. Molec. Plant-Microbe Interactions 24:458-468.

1. A recombinant nucleic acid molecule comprising a nucleic acid sequence comprising at least 15, 16, 17, 18, 19, 20, 21, 22, 23, 24 or 25 contiguous nucleotides that are complementary to SEQ ID NO: 1, SEQ ID NO: 3, SEQ ID NO: 6, SEQ ID NO: 7 or SEQ ID NO: 8, wherein transcription of the nucleic acid molecule or the presence of the nucleic acid molecule reduces expression of a succinic semialdehyde dehydrogenase (SSADH) gene in a plant cell.

2. The recombinant nucleic acid molecule of claim 1, wherein the nucleic acid sequence is a DNA molecule, and the DNA molecule is operably linked to a promoter.

3. The recombinant nucleic acid molecule of claim 2, wherein the promoter is a heterologous promoter.

4. The recombinant nucleic acid molecule of claim 1, wherein the nucleic acid sequence is a DNA sequence, and wherein transcription of the nucleic acid sequence produces an interfering RNA molecule targeting the SSADH gene, wherein the interfering RNA is a double-stranded RNA or antisense RNA, one strand of which comprises at least 15, 16, 17, 18, 19, 20, 21, 22, 23, 24 or 25 contiguous nucleotides that are complementary to SEQ ID NO: 1, SEQ ID NO: 3, SEQ ID NO: 6, SEQ ID NO: 7 or SEQ ID NO: 8.

5. The recombinant nucleic acid molecule of claim 1, wherein the nucleic acid sequence is an RNA molecule.

6. The recombinant nucleic acid molecule of claim 5, wherein the nucleic acid sequence is a dsRNA or siRNA.

7. The recombinant nucleic acid molecule of claim 1, wherein the nucleic acid sequence further comprises an DNA sequence encoding movement leader sequence, or a corresponding RNA sequence.

8. The recombinant nucleic acid molecule of claim 7, the movement leader sequence comprises at least 18 contiguous nucleotides of SEQ ID NO: 5.

9. The recombinant nucleic acid molecule of claim 1, wherein the nucleic acid molecule is operably linked to a GUS loop sequence or an intron sequence.

10. A recombinant vector comprising or encoding the recombinant nucleic acid molecule of claim 1.

11. A transgenic plant cell, a transgenic plant part, or a transgenic plant comprising the nucleic acid molecule of claim 1.

12. A plant comprising a nontransgenic grafted scion, and a transgenic rootstock, wherein the rootstock comprises the recombinant nucleic acid molecule of claim 1.

13. A composition comprising the recombinant nucleic acid molecule of claim 1.

14. The composition of claim 13, wherein the recombinant nucleic acid molecule is an RNA molecule, and the composition is for delivering the RNA molecule into phloem cells of a plant.

15. The composition of claim 14, wherein the composition is in a nanoemulsion formulation or an aqueous formulation.

16. A method of repressing, preventing or otherwise reducing a bacterial or fungal infection of a plant, comprising expressing the recombinant nucleic acid sequence of claim 1 in the plant, or applying the recombinant nucleic acid sequence to the plant.

17. The method of claim 16, wherein said plant is a citrus tree.

18. The method of claim 16, wherein said infection is caused by a biotrophic plant pathogen.

19. The method of claim 16, wherein said infection is caused by a Liberibacter.

20. The method of claim 16 wherein said infection is caused by a Liberibacter infecting citrus.

21. The method of claim 16, wherein said infection is caused by Ca. Liberibacter asiaticus (Las).

22. A method of repressing, preventing or otherwise reducing a bacterial or fungal infection of a plant, comprising topical application, injection, soil drench, root feeding, fertilization, mechanical etching or any form of introduction of the nucleic acid of claim 1 to said plant.

23. The method of claim 22, wherein the topical application utilizes lecithin and or gelatin formed into an emulsion.

24. The method of claim 22, wherein said plant is a citrus tree.

25. The method of claim 22, wherein said infection is caused by a biotrophic plant pathogen.

26. The method of claim 22, wherein said infection is caused by a Liberibacter.

27. The method of claim 26, wherein said infection is caused by a Liberibacter infecting citrus.

28. The method of claim 26, wherein said infection is caused by Ca. Liberibacter asiaticus (Las).

29. The method of claim 22, wherein the topical application comprises spraying.

30. The method of claim 29, wherein the spraying comprises spraying an aqueous formulation.
